(19) United States
(12) Patent Application Publication
(10) Pub. No.: US 2005/0197614 A1
(43) Pub. Date: Sep. 8, 2005

(54) Occlusive biomedical devices, punctum plugs, and methods of use thereof

(76) Inventors: Wilson Pritchard, Memphis, TN (US); Cedric Flowers, Bartlett, TN (US); Tony Prescott, Arlington, TN (US); Rick Mendius, Collierville, TN (US); Clive Hallam, Memphis, TN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/071,985

(22) Filed: Mar. 4, 2005

Related U.S. Application Data
(60) Provisional application No. 60/550,132, filed on Mar. 4, 2004.
(60) Provisional application No. 60/557,368, filed on Mar. 29, 2004.
(60) Provisional application No. 60/564,858, filed on Apr. 23, 2004.
(60) Provisional application No. 60/637,569, filed on Dec. 20, 2004.

(51) Int. Cl. A61M 5/00

(52) U.S. Cl. 604/8

(57) ABSTRACT

Certain embodiments include a punctum plug for blocking flow of lacrimal fluid in an eye, the plug having an introducible portion comprising a dehydrated material hydratable by physiological saline to swell from a first diameter to a second diameter that is at least 50% greater than the first diameter, wherein the portion is swellable by the lacrimal fluid to occlude the punctal opening to block the flow of the lacrimal fluid through the punctal opening, wherein the dehydratable material degrades in less than about seven days in the punctal opening of the patient. Certain embodiments include a device for occluding a nasolacrimal passage, the device comprising an introducible portion made with an anisotropically swellable material. Some embodiments are punctum plugs made with a polysaccharide in the group consisting of gellan, welan, S-88, S-198 and rhamsan gum.

### RELATED APPLICATIONS

This application claims priority to U.S. patent application Ser. Nos. 60/550,132 filed Mar. 4, 2004, 60/557,368 filed Mar. 29, 2004, 60/564,858 filed Apr. 23, 2004, and 60/637,569 filed Dec. 20, 2004, each of which are hereby incorporated by reference herein. This application is also related to U.S. patent application Ser. No. ______, entitled MATERIALS FOR MEDICAL IMPLANTS AND OCCLUSIVE DEVICES, filed Mar. 3, 2005.

### FIELD OF USE

The field of use is related to occlusive devices, and includes disclosure of nasolacrimal occlusive devices such as canalicular plugs placed into the punctal opening of the lacrimal duct.

### BACKGROUND

A variety of eye problems are related to an insufficient volume of tears on the surface of the eyes. The most common is keratoconjunctivitis sicca, also known as dry eyes. Contact lens problems are also often provoked by a lack of tear volume. A common cause for the insufficient tear volume is the drainage of tear fluid through the punctal opening of the lacrimal duct and into the nasal passage, thereby removing the fluid from where it is needed at the eye surface. Furthermore, drainage of tear fluid through the lacrimal duct into the nasal passage is believed to be the cause of or associated with several additional problems such as post nasal drip, sinusitis, allergies, headaches, and snoring.

A number of methods for closing the punctal opening have been used to prevent drainage of tears through the lacrimal duct, including suturing, laser sealing, and plugging. Plugging with a canalicular plug, such as a punctum plug or a lacrimal plug, is relatively inexpensive, and is being performed with increasing frequency.

### SUMMARY

Despite significant progress in these arts, there continues to be a need for nasolacrimal devices that degrade at a controlled rate, that are easily removed, and/or which fit more comfortably and securely, especially in light of the fact that the distribution of canalicular sizes ranges significantly among patients. These and other needs are addressed herein by inventive embodiments that include nasolacrimal devices that are swellable, anisotropically swellable, chelation resistant, controllably degradable, triggerably degradable, gellable by physiological fluids, or made with foam.

Some embodiments are materials and methods related to an occlusive device such as a punctum plug for blocking flow of lacrimal fluid in an eye. These embodiments may have an introducible portion of the plug sized for introduction into a punctal opening of the eye, the portion comprising a dehydrated material hydratable by physiological saline to swell from a first diameter to a second diameter that is at least 50% greater than the first diameter, wherein the portion is swellable by the lacrimal fluid to occlude the punctal opening to block the flow of the lacrimal fluid through the punctal opening, wherein the dehydratable material degrades in less than about seven days in the punctal opening of the patient.

Some embodiments are materials and methods for occluding a nasolacrimal passage. Such embodiments may include a device comprising an introducible portion that is introducible into the nasolacrimal passage to at least partially block movement of a fluid through the passage, wherein the introducible portion comprises an anisotropically swellable material that anisotropically swells in vitro in a physiological saline solution when not subjected to constraining forces. In some other embodiments, at least a part of the introducible portion comprises at least one polysaccharide in the group consisting of gellan, welan, S-88, S-198 and rhamsan gum.

Some embodiments are nasolacrimal occlusive devices made of swellable materials. A controlled amount of swelling can be useful to set the implant in place, but too much swelling can harm surrounding tissue. A tissue is a solid or partially solid portion of a patient's body. Tissues that surround a preexisting or created space in a body define that space, e.g., the walls of an artery define the artery lumen, and the tissue around a bolus of material injected into a muscle defines the space thereby created. In some circumstances, the implant must be firmly set into an opening in a patient so that a relatively high degree of swelling is desirable, but the high degree of swelling tends to push the implant out of the opening so that the implant is not stable. Accordingly, controllably swellable materials may be used, as described, below.

Other embodiments are provided that have a combination of some or all of the above-described features, or have various other advantages or features that contributes to the improvement of these arts.

### BRIEF DESCRIPTION OF THE DRAWINGS

FIG. 1 is a representation of the anatomy of the human eye and associated lacrimal excretory system.

FIG. 2A is a plan view, with representative dimensions, of one embodiment of a punctal plug in accordance with the present invention.

FIG. 2B is a plan view, with representative dimensions, of a second embodiment of a punctal plug.

FIG. 3 is an enlarged view of a detail of the eye anatomy showing the punctal plug embodiment of FIG. 2B in place in the lower punctal opening.

FIG. 3A is a sectional view taken along line 3A-3A in FIG. 3.

FIG. 4 is a plan view of a dilator tool for use in enlarging the punctum and associated canaliculus prior to receiving the punctal plug.

FIG. 5 is a plan view of an inserter tool for grasping, manipulating and inserting the plug into the punctal opening.

FIG. 5A is an enlarged view showing the detail of the head portion of the dilator tool of FIG. 5 grasping the punctum plug embodiment of FIG. 2B prior to insertion.

FIG. 6A shows a high acyl form of gellan gum.

FIG. 6B shows a low acyl form of gellan gum.

FIGS. 7A and 7B are diagrams showing a nasolacrimal occlusion device that swells after contact with a tear or other physiological fluid.

FIG. 8 depicts Schirmer data collected for another embodiment of an occlusive device.

### DETAILED DESCRIPTION

Various materials and methods for making improved nasolacrimal occlusive devices are described herein. Certain embodiments are directed to nasolacrimal devices that are swellable, anisotropically swellable, chelation resistant, controllably degradable, triggerably degradable, and gellable by physiological fluids. Embodiments include swellable devices that expand in volume in response to lacrimal fluid. And other embodiments are anisotropically swellable devices that are swellable in a canaliculus to expand radially, but not longitudinally, whereby the device fits securely without being dislodged by longitudinal extension. And certain devices described herein are degradable at a predetermined rate by virtue of materials that are incorporated into their structure. Also disclosed are devices made of materials that are degradable upon exposure to a triggering substance that causes degradation. Other devices and materials are also disclosed, including plugs made from expandable foam and compositions that gel upon exposure to physiological fluids.

Resistance to chelation may be advantageous for nasolacrimal occlusive devices that are exposed to chelating agents, which are commonly found in some ophthalmic solutions, e.g., contact lens solutions. Accordingly, some embodiments describe chelation-resistant implantable materials, including materials that are degradable over short term, degradable over a long term, or effectively undegradable.

While some conditions are best treated with permanent or nondegradable plugs, the use of temporary plugs can be beneficial in some situations. For instance, temporary punctal or canalicular occlusion may be used as a diagnostic aid to determine the potential effectiveness of permanent occlusion. Temporary occlusion may also be used in the treatment of dry eye syndrome and the dry eye components of various ocular surface diseases such as corneal ulcers, conjunctivitis, pterygium, blepharitis, keratitis, red lid margins, recurrent chalazions, recurrent corneal erosion, filamentary keratitis, acquired abnormalities, and other external eye diseases. Temporary occlusion may also benefit patients experiencing symptoms such as redness, burning, reflex tearing, itching or foreign body sensations which can be relieved by blockage of the canaliculus. In addition, temporary occlusion may be useful in decreasing contact lens intolerance, to evaluate treatment of ocular dryness secondary to contact lens use, for increasing retention/enhancement of ocular medications or lubricants on the eye, for maintenance of ocular flora, punctal stenosis, and to enhance healing and comfort after surgery.

The use of the punctum plug in the treatment of dry eye conditions may be related to conditions in which the volume of aqueous tears is markedly decreased on a chronic basis. In such a condition, lid movement becomes scratchy or painful because of inadequate aqueous lubrication between the inner lid edge and the corneal surface; the exposed corneal cells lose water to the atmosphere and become desiccated (with associated pain and cell damage). Cell injury or death can be detected by the use of certain dyes such as Rose Bengal or Fluorescein. In severe cases, untreated dry eyes can become infected, ulcerated or blind.

The decrease of aqueous tears can result from a variety of factors such as age, disease states, injury to the lacrimal gland tissue as well as the side effects of use of certain drugs. As stated, a significant portion of the surface tear volume is contained in the upper and lower menisci, which are in hydraulic contact with the punctal openings. Reportedly, during the blink, there is an outflow of tears via the lacrimal system. Occlusion of one or both of these puncta should decrease or stop fluid loss by this route and benefit eyes which are diseased because of aqueous deficiency.

Occlusion of the punctum for treatment of dry eye syndrome and the dry eye components of various ocular surface diseases has a long clinical history. It was first described over thirty years ago. Devices used in these non-surgical procedures were commonly made from animal derived collagen, absorbable polyglyconate suture material, thermosensitive hydrophobic acrylic polymer, and silicone. With the exception of new materials, characterized as biocompatible, and specifically safe for use in the eye, significant design changes have not been made over the past decade. Various embodiments are described herein that improve these arts for the benefit of patients.

Nasolacrimal Occlusion Devices

Some punctal plug occlusion devices are meant to be inserted below the punctal opening and others possess a rim meant to sit atop the punctal opening. Devices of both categories can be fabricated using hydrogels and other materials as described herein.

Devices inserted below the punctal opening are referred to herein as subpunctal devices. Advantages to this type of device include ease of insertion and low cost. Subpunctal devices are simple in design, being cylindrical pieces of material with dimensions of, e.g., about 1.5 to about 2 mm in length and about 0.3 to about 0.4 mm in diameter, or other sizes as appropriate for a patient. A disadvantage of certain subpunctal devices is potential difficulty in removal should the plug no longer be needed.

Devices made with a rim which rests atop the punctal opening provide some advantage in that they can be easily visualized and are simple to remove. A rimmed plug that incorporates a hydrogel should have a resistance to cutting, or cutting strength, that allows removal with forceps. Parts outside the punctum should maintain constant or near-constant dimensions over time and in response to changes normally encountered during use. Removal of rimmed punctum plugs is usually accomplished by seizing the plug below its rim with forceps and pulling. This removal method presents a challenge for hydrogel materials as their cutting strength is normally poor so that a hard object such as forceps will cut through the hydrogel. To address this issue, the topmost parts of the plug may be made from materials other than a hydrogel.

Various strategies may be used for removal of rimmed or subpunctal devices, including physical removal, flushing the lachrymal system, timed dissolution/degradation, and triggerable disintegration caused by exposure to chemicals. Physical or surgical removal may be, for example, by use of forceps, which can be a particular challenge for subpunctal devices. Flushing of the lachrymal system may be effective, especially if the solution used in flushing is capable of either solubilizing or decreasing dimensions of the occlusive material. Timed dissolution is helpful for temporary occlusion, but alternative removal methods may be used if the material needs to be removed before resorption has occurred.

Chemically triggerable disintegration should require a minimum of time and effort to accomplish the removal. Many ocular medications contain chelating agents which patients may apply with varying frequency so that an occlusive device made from chelation-sensitive materials would be suboptimal.

A variety of nasolacrimal occlusive devices may be used to at least partially fill and to at least partially block movement of a fluid through a nasolacrimal passage. A general description of some aspects of such structures is provided herein. Certain dimensions and procedures are provided for exemplary purposes, but are not intended to limit the scope or spirit of the invention.

Referring to FIG. 1, there is shown a representation of the human eye anatomy and the associated lacrimal excretory system. For purposes of the present discussion it will be sufficient to focus on the latter which consists of the upper and lower lacrimal ducts 10 and 12, better known as the canaliculae, and the tear or lacrimal sac 14. The upper and lower canaliculae 10, 12 each terminate in respective small punctal apertures 11 and 13 situated on a slight elevation at the medial end of the lid margin at the junction 15 of the ciliary and lacrimal portions about 6 mm from the medial canthus 17. The punctal apertures are round or slightly ovoid openings approximately 0.3 mm in size and surrounded by a fairly dense, relatively avascular connective ring of tissue about 1 mm in depth. Each of the punctal openings 11, 13 leads into a vertical portion 10a, 12a of the respective canaliculus, which is about 2.5 to 3.5 mm in length, before turning horizontally for about 8 mm to join its other canaliculus at the entrance of a lacrimal sac 14. The canaliculae 10, 12 are tubular about 0.5 mm in diameter and lined by stratified squamous epithelium surrounded by elastic tissue which permits the canaliculus to be readily dilated to three times normal size.

In the treatment of keratoconjunctivitis sicca and other ophthalmic ailments where it is desired to prevent or decrease the drainage of lacrimal fluid and/or medication from the eye, the punctal aperture in either or both of the upper and lower lids are may be blocked by a removable plug member 20, two respective embodiments of which are shown in FIGS. 2A and 2B. Referring initially to the embodiment of FIG. 2A, the punctum plug 20 has an axial length of approximately 3.2 mm and consists of three portions; a projecting tip or barb portion 22, a middle neck or waist portion 24 of somewhat smaller diameter than the tip, and a smooth disc-like head portion 26 of relatively larger diameter. The plug embodiment 20′ of FIG. 2B is of generally similar dimensions to the first-described embodiment with a somewhat blunted tip or barb portion 22′, a cylindrical middle portion 24′ of substantially the same dimension, and a dome-shaped head portion 26′ of somewhat smaller diameter than its counterpart in the embodiment of FIG. 2A. The head portion 26, 26′ of both embodiments may be provided, if desired as an alternative to grasping it with forceps, with a central bore opening 28, 28′ adapted to receive the projecting tip of an inserter tool to provide a releasable grip on the plug as it is manipulated for insertion.

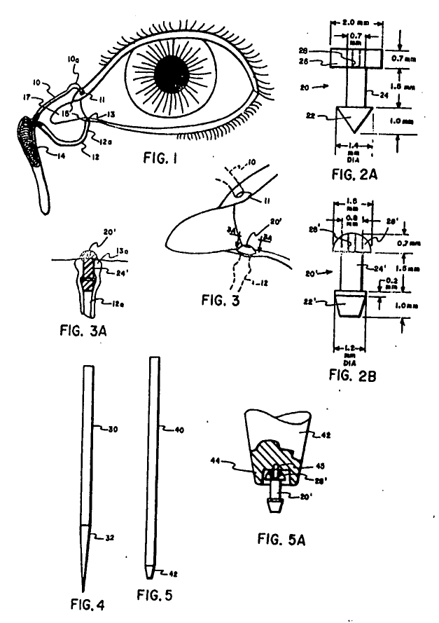

The projecting tip or barb portion 22, 22′ of the respective embodiments of the punctum plug is designed with either a tapered 22a or semi-tapered tip 22a′ for further dilation and ease of insertion into the punctal opening. The tip portion 22, 22′ is flared back to a somewhat larger base 22b, 22b′, typically 1.2-1.4 mm in diameter, and then narrows down to a waist or neck portion 24, 24′ of a somewhat smaller diameter, typically 0.7-0.8 mm. The distended vertical canaliculus 12a and the punctal sphincter ring 13a (FIG. 3A) tightens upon the respective tip and waist portions of the plug to firmly secure it from accidental extrusion. The head portion 26, 26′ of the respective plug embodiments is sufficiently large, approximately 1.5-2.0 mm in diameter, as it rests on the punctal opening so as to prevent the plug from passing down into the canaliculus. The plug head is very smooth and of disc or dome shape which allows it to rest in the lacrimal lake and against conjunctivae and cornea with very little resultant irritation.

In certain embodiments of the invention the plugs 20, 20′, particularly the head portion 28, 28′, may be of medication-impregnable porous material such as HEMA hydrophilic polymer, or may be otherwise adapted as with capillaries or the like, to store and slowly dispense ophthalmic drugs to the eye as they are leached out by the lacrimal fluids.

An exemplary technique for inserting the plug into the punctal aperture and associated canaliculus will now be set forth. The affected eye is first anesthetized with a topical anesthetic such as Properacaine, then a shortened cotton-tipped applicator is soaked in the same or similar topical anesthetic and put into the medial canthal area at the juncture of the upper and lower lid for 5 to 10 minutes. Next a punctum dilator 30, which as shown in FIG. 4 is in the form of an elongated rod of Teflon polytetrafluorethylene material terminating in a tapered awl-like flexible tip portion 32, is carefully used to slowly dilate the punctum and associated vertical canaliculus to about 2½ to 3 times its normal size, or about 1.2 mm, taking care to avoid breaking of the punctal connective tissue ring which, if it occurs, would produce until healed a looser, sloppier fit of the plug and possible accidental extrusion thereof.

The plug itself is placed in the punctal opening with conventional forceps or with the aid of a special inserter tool 40 which, as shown in FIGS. 5 and 5A, is in the form of a pencil-like rod terminating in a blunted head 42 provided with a recessed central portion 44 of slightly larger diameter and deeper than the head portion 28, 28′ of the respective punctum plugs 20, 20′. To temporarily engage the plug by its head portion, a thin finger member 45 projects outwardly from the center of the recess and is adapted to mate with a corresponding bore 28, 28′ in the head of the plug. The friction fit is sufficiently tight between the projecting finger 45 and the mating bore hole 28, 28′ that the plug is securely held by the inserter tool 40 as it is manipulated into the punctal aperture. As previously mentioned, the tip or barb portion 22, 22′ of the plug may be pointed, or at least partially so, to encourage some further dilation of the punctum and the canaliculus as the plug is inserted therein.

The plug is advanced into the depth of the canaliculus by manipulation of the inserter tool until the head portion 26, 26′ is seated on the punctal opening. Thereupon, a simple shearing or wobbling motion of the inserter tool springs the projecting finger 45 from the plug head, permitting disengagement and removal of the tool leaving the punctum plug inserted in place. Following insertion the patient will usually experience some transient discomfort which can be relieved by aspirin or similar analgesic.

When it is desired to remove the plug, the head portion 26, 26′ of the plug, or the neck 24, 24′ just under the head, may be grasped with forceps and the plug withdrawn from the punctal opening. If necessary, topical anesthetic can be applied for the removal technique in which case, as an alternative or in addition to the use of forceps, the plug may be squeezed out of the punctal opening by pressure applied to the horizontal portion of the canaliculus, accompanied by movement toward the punctal opening.

Other features may be incorporated into a nasolacrimal occlusive device, as set forth elsewhere herein. These various features may be combined with the various materials and methods set forth and referenced herein. For example, the shaft further may have a ridge or a collapsible portion. The device, or a portion thereof, may further comprise a degradable portion. The device, or a portion thereof, may further comprise a therapeutic agent with/without dimethlysulfoxide (DMSO) and/or methyl-sulfonyl-methane (MSM). The device may be graspable by standard forceps for insertion into a punctum. Degradation of a material is a process that causes a material to lose its mechanical properties, e.g., its strength, cohesiveness, or resiliency. Degradation may occur by a variety of mechanisms, e.g., hydrolysis of chemical bonds, dissociation of ions that crosslink polymers that form the material, or a host-response to the material after its implantation into the host. In some instance, an implanted material is referred to as being fully degraded or dissolved, meaning that it has degraded to the point that the implanted material is essentially no longer visible at the implant site; such a process may occur by any of a variety of degradation mechanisms. Full degradation or dissolution may be modeled in a laboratory by maintaining a material in a container at physiological temperate, pH, and osmotic pressure until it is no longer visible to the naked eye.

Other patents and patent applications set forth further aspects, structures, methods of use, and details of punctum plugs, nasolacrimal occlusive devices, and related items. Incorporated by reference herein are U.S. Provisional Application Nos. 60/550,132, entitled “Punctum Plugs, Materials, And Devices”, 60/564,858, entitled “Nasolacrimal Occlusive Devices and Methods of Use”, 60/637,569, entitled “Occlusive Biomedical Devices and Methods of Use Therefor” and U.S. Pat. Nos. 6,629,533; 6,605,108; 6,344,047; 6,306,114; 6,1743,21; 6,082,362; 6,027,470; 5,980,863; 5,951,565; 5,921,990; 5,830,226; 5,741,292; 5,524,357; 5,334,137; and 5,283,063 all of which are hereby incorporated by reference herein. A variety of materials may advantageously be employed in the construction of a nasolacrimal occlusive device. Some such materials are set forth in detail in U.S. patent application Ser. No. 60/557,368 entitled “Chelation Resistant And Anisotropically Swelling Materials For Medical Implants And Occlusive Devices”, hereby incorporated by reference herein.

Gellan, Depolymerized Gellan, and Related Polysaccharides for Biomedical Uses

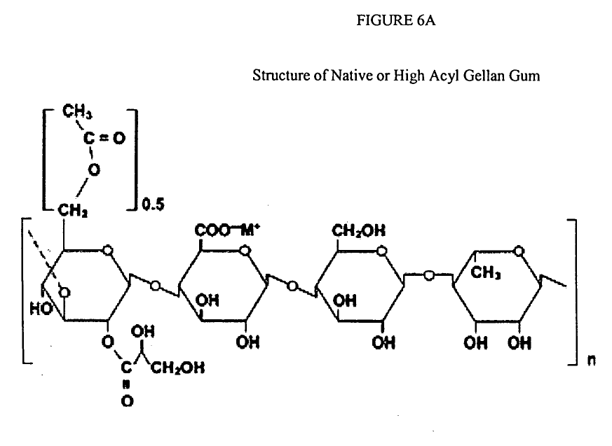

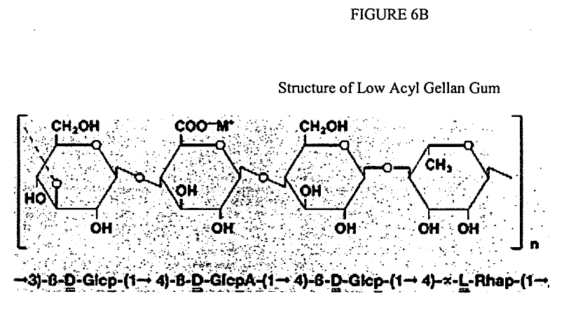

Biomedical devices may be made using gellan, depolymerized gellan, and related polysaccharides. As set forth in greater detail in U.S. patent application Ser. No. 60/557,368, gellan gum is a polysaccharide, and is prepared commercially as a bacterial exopolysaccharide using fermentation, e.g., from Sphingomonas elodea (previously called Pseudomonas elodea). FIG. 6 shows the structure of a form of gellan. The properties of a gellan-based material depend, in part, on the degree of gellan's acylation and the ions present. If left acylated, gellan tends to form soft, elastic, transparent and flexible gels. When de-acylated it forms hard, relatively non-elastic brittle gels. A gellan gum solution may hold particles in suspension without significantly increasing the solution's viscosity. A gel sol transition occurs at about 50° C. dependent on concentration. Thermoreversible gels form on cooling in the presence of cations even at low (0.1% w/w) to very low (0.005% w/w) concentrations of gellan. Gellan can be formulated at concentrations and conditions so that it gels in response to exposure to physiological conditions.

Gellan and related materials may be prepared as already described, e.g., in U.S. patent application Ser. No. 60/557,368, and made into a device for occluding a nasolacrimal passage as described herein, or as referenced herein. Gellan is an anionic polysaccharide that gels in the presence of cations such as Sodium (Na⁺) and Calcium (Ca⁺⁺). It is soluble in water, and hydrates rapidly in solution. As the gel hydrates, it also expands (up to 500% or more depending on the concentration of gellan and the strength of the ionic bonds). After hydration, the gellan becomes pliable and malleable to conform to the inside of the volume that constrains it (assuming the volume is less than or equal to the physical size of the gel in its hydrated state).

Gellan has a long history of clinical use in humans that spans 15 years. It has been studied as a drug delivery material because of its in situ gelling properties. It has also been studied as a time release material for drug delivery for its controllable and predictable dissolution properties (as a gel) in contact with mucosal membrane (analogous to the punctum) in vivo, and for insulin delivery in vivo. And gellan has been studied for both its gelling properties and dissolution rate. Several studies have been completed dealing with the safety of gellan for use in the eye. And more specifically, numerous studies involving gellan as a safe and efficacious delivery vehicle for TIMOLOL (antiglaucomatous medication) have been completed

Polysaccharides closely related to gellan are those such as welan, S-88, S-198 or rhamsan gums; these can also be processed by the methods described herein, and can be used as substitutes for, or added to, gellan gum. Other polysaccharides related to gellan are alginate, curdlan, carboxymethylcellulose, crosscarmellose, poly(acrylic acid), xanthan, carrageenan, carboxymethyl chitosan, hydroxypropyl carboxymethyl cellulose, pectin, gum Arabic, karaya gum, psyllium seed gum, carboxymethyl guar, and mesquite gum; methods described herein can be generally adapted for use with these polysaccharides.

As described in greater detail below, some embodiments are materials and devices that resist degradation, resist chelation, and are at least partially made of gellan. Sodium gellan is unaffected by disodium EDTA, a chelating agent. Disodium EDTA can exchange its sodium ions for crosslinking ions in a given ionically-crosslinked hydrogel. Unlike many other ionic, gelling polymers such as sodium alginate, sodium gellan remains a gel in vivo. Hence removal of divalent or trivalent ions and conversion to sodium gellan does not affect the physical state of the hydrogel. Gels strong enough to be used as implantable plugs may be dense and, to that end, may be processed from at least 5% gellan gum in water or DMSO. Other concentrations include between 1% and 50%, including 5%-15%, and 15%; persons of ordinary skill in these arts will appreciate that all values and ranges within the explicit limits are contemplated. Gellan will not normally resorb or dissolve after implantation into a patient, but can be removed by exposure to salt-free water.

Swellable Materials and Devices

As a dry gel material hydrates, it typically swells to fill a space and then takes up no more water. For example, if a dry gel material is placed in thin walled flexible silicone tubing and then hydrated, the gel will swell to fill, but only slightly deform, the tubing. A hydrogel plug that incorporates an unconstrained hydrogel material will thus be more successful in swelling to achieve a secure fit. This unconstrained hydrogel material may be located at, e.g., the bottom or nose of a plug. The top end of a plug, the neck and rim, may include a strong, non-swelling material to address the issues of cutting strength and dimensional stability. For example, a nonswelling plastic may be used to cover the upper portion of a polysaccharide plug so that the polysaccharide will swell against the plastic but not further expand. The other portion of such a plug, however, will be free to swell. A punctum plug may be shaped to have a configuration as shown in, e.g., FIGS. 2-3.

When a swellable material's expansion is limited by a constraining tissue, the material exerts a force against that tissue. Swellable means something that can be swollen in response to a fluid. Some hydrogels are swellable because they are less than fully hydrated when introduced into a patient, so that the hydrogel imbibes fluid from the patient. Such hydrogels may be, e.g., desiccated, lyophilized, or hydrated but not fully hydrated. A hydrogel that has been dehydrated to remove water is referred to herein as a hydrogel. Hydrogels do not dissolve in solution. Certain materials that are specially prepared to dissolve or otherwise break up in substantially deionized water, but not physiological solutions, are referred to herein as hydrogels since they are chemically crosslinked and do not dissipate under the conditions of their intended use prior to their intentional removal with deionized water. Substantially deionized water is water with no ions, or with a low concentration of ions, e.g., less than about 50 milliOsmoles, or less than about 10 milliOsmoles.

Gellan, polysaccharides closely related to gellan, and other polysaccharides related to gellan may be used to make swellable occlusive devices, e.g., punctum plugs. Swelling of a polysaccharide may be, for example, between 25% and 1000% as measured in a physiological solution without restriction. Swellable plugs may be made with essentially randomly oriented polymers so that there is no preferential direction of swelling in the polysaccharide portion of the plug.

Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were dehydrated in a graded alcohol series to 91% alcohol and either stretched to twice their original length or left unstretched. They were allowed to air dry. Prototype occlusive devices were fabricated by cutting neutralized extrusions into cylindrical pieces. Their dry dimensions were 1.524 millimeters in length and 0.254 millimeters in diameter for stretched extrusions and 0.762 millimeters for unstretched extrusions. Once placed into physiological saline and allowed to swell to their maximum extent, stretched extrusions shrank to 1.27 millimeters in length and swelled to 1.016 millimeters in diameter. This represents a 16.6% decrease in length and a 300% increase in diameter. Unstretched extrusions swelled to 2.54 millimeters in length and 1.27 millimeters in diameter. This represents a 166% increase in both length and diameter. The measurements were made using a scale marked in increments of 0.01 inches, which were then converted to metric units.

Anisotropically Swelling Materials and Devices

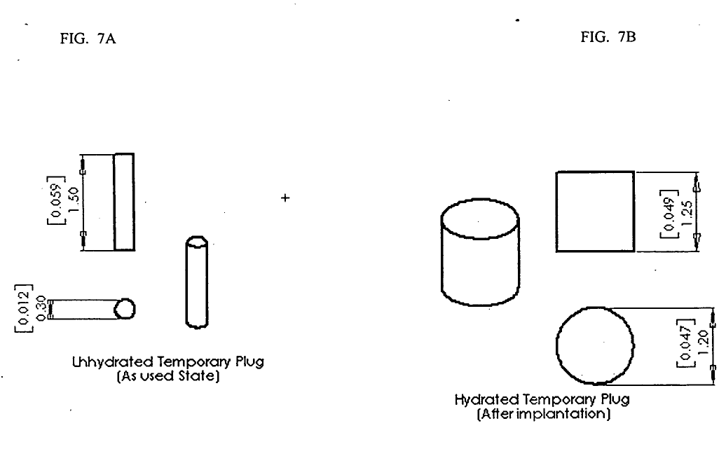

A swellable occlusive device placed into a lumen or opening can sometimes be forced out of the opening by the swelling process. Or a portion outside the opening can swell to make appropriate placement difficult. It is therefore helpful in some situations to use a device which swells only in lateral dimensions, thus effectively blocking, but not protruding from, the opening, e.g., a duct or canal. Further, the device may shrink in at least one dimension, such that a thin, cylindrical device becomes short and fat once hydrated. Punctum plugs, for example, may be made with anisotropically swelling materials. FIGS. 7A-7B depicts an example of a swellable punctum plug, and indicates dimensions before and after swelling. The dimensions in the Figures are based on actual results but are exemplary only, and may be suitably modified in light of the material used and the properties of the lumen or canaliculus that receives it.

An anisotropically swellable material does not swell equally in all directions. When unrestrained, such materials swell differentially. For example, an anisotropically swellable hydrogel may swell only in one or two directions while maintaining or diminishing in another direction. When restrained, such materials apply a greater force in the direction in which they preferentially swell. An anisotropically swellable polymer material may be prepared by aligning polymer molecules in one or more preferential directions. Polymer molecules are arranged randomly tend to move apart in all directions upon hydration, and thus demonstrate isotropic swelling (essentially the same in all directions). If polymer molecules are aligned parallel to each other, however, they move apart in only one or two dimensions, as they are (ideally) already fully extended in a third. Upon hydration, molecularly aligned hydrogels would demonstrate anisotropic expansion. Some anisotropic materials comprise polymers that are substantially parallel to each other in their molecular orientation, with the material having enough such polymers so that its macroscopic swelling properties are affected. Hydration, in its strictest sense, refers to a process involving water, but other liquids can also serve to accomplish the swelling of polymers, and such processes are contemplated herein. In some embodiments, hydrogels are fabricated by crosslinking of water-soluble polymers so that the crosslinking is only extensive enough to insolublize the material in water. Upon hydration, the oriented polymer molecules are forced apart, held together only by crosslinks.

Anisotropically swellable materials may be prepared as described, below, or as already described, e.g., as in U.S. patent application Ser. No. 60/557,368 or 60/637,569, and made into a device for occluding a nasolacrimal passage as described herein. The device may include an introducible portion that is introducible into a nasolacrimal passage, wherein at least a part of the introducible portion comprises an anisotropically swellable material that anisotropically swells in vitro in a physiological saline solution when not subjected to constraining forces. A nasolacrimal passage refers to a portion of the lacrimal excretory system. A physiological saline refers to a solution having a pH in a physiological range, e.g., in a range of about 7.0 to about 7.4 and an osmolarity in a physiological range, e.g., between about 300 and about 330 milliOsmoles. Phosphate buffering systems, and others, are known for making physiological salines.

A material may be tested for anisotropic swelling by measuring a sample's dimensions before and after exposure to a large excess of physiological saline, with final measurements being conducted when the swelling of the material has essentially ceased. In the case of a plug, the plug's dimensions could be measured in a state that is equivalent to its conditions immediately prior to insertion into a patient, and after exposure to the physiological saline in vitro. Unless stated otherwise, reported swelling measurements are made at room temperature (about 20° C.), but degradation in physiological saline is discussed in the context of physiological temperatures (37° C.).

Use of anisotropic hydrogels as materials for punctal occlusion solves a problem with many devices. The size of the punctal opening varies among patients; therefore the punctum must be measured, and a properly sized plug inserted. Devices made from anisotropic hydrogels, however, require neither measuring punctal size nor keeping of an inventory of many differently sized punctum plugs. Proper dimensions necessary for punctal occlusion are achieved through hydration of the device. For example, the device will swell radially until it has expanded sufficiently to occlude the nasolacrimal passage but will otherwise change its other dimensions in a controlled manner.

An anisotropically swellable nasolacrimal occlusive device may further include a volume, a first length and a second length perpendicular to the first length, wherein exposure to physiological fluid causes the volume to increase, the first length to undergo a first percentage increase and the second length to undergo a second percentage increase that is less than the first percentage increase for the first length. Examples of such increases, for the first or the second percentage increase, include at least about 25%, at least about 100%, at least 300%, and between about 10% and about 500%; persons of ordinary skill in these arts will immediately appreciate that all ranges and values within these explicitly set forth ranges are contemplated. Further, the second percentage increase may be, e.g., less than 100%, less than 50%, or less than 0% (i.e., shrinking), and between −50% (i.e., shrinking by one-half) and 100%; persons of ordinary skill in these arts will immediately appreciate that all ranges and values within these explicitly set forth ranges are contemplated. Referring to FIGS. 2A and 2B, for example, plug 20 may be made of an anisotropically swellable material having polymeric alignment parallel to the longitudinal axis, with the second length being about 3.2 mm before swelling and the first length being about 0.7 or 0.8 mm before swelling. Thus, for example, the portion 24, 24′ would swell against a wall of a nasolacrimal passage after the device was inserted into the same.

A nasolacrimal device may be made entirely of an anisotropically swelling material, or only partially. For example, referring to FIG. 2A, waist or neck portion 24 could be anisotropically swellable while head portion 26 was not. One option for manufacturing a device would be to provide the nasolacrimal occlusive device in component parts that are assembled by a user immediately prior to use. For example, head portion 26 could be provided with an opening for receiving waist or neck portion 24. A user would then fit portion 24 into the head prior to use.

Another embodiment is a device for occluding a nasolacrimal passage, the device comprising an introducible portion that is introducible into the nasolacrimal passage to at least partially block movement of a fluid through the passage, wherein at least a part of the introducible portion comprises a length and a swellable material that swells after introduction into the nasolacrimal passage to essentially occlude the passage while the swelling causes the length to increase by less than about 10%, 25%, or 0%.

In general, an anisotropically swellable nasolacrimal occlusive device may be made from suitable polymers aligned in a predominantly parallel orientation relative to each other. Aligning the polymers may comprise at least one technique chosen from the group consisting of spin coating, spray coating, stretching, unidirectional freezing, extrusion from liquid crystalline solution, ordered convection, and stretching plus drying of an extrusion. A molecularly oriented occlusive device of cylindrical shape can be made in these ways, but the simplest and preferred method is usually by stretching and drying of an extrusion. In certain embodiments, aligning the polymers may comprise stretching the material and soaking the material in a fluid comprising a mineral or organic acid before stretching the material. And aligning the polymers may comprise acidification of an anionic polymers before dissolution in DMSO. Examples of materials include sodium gellan, carboxymethylcellulose sodium, calcium alginate, and calcium gellan.

Monofilaments of a hydrogel material may be made, e.g., by extrusion and subsequent stretching to at least 1.5-2 times their original length. Upon drying, they can be cut into small cylinders for easy insertion into a duct or canal. For occlusion of the lachrymal system, these devices are typically 1.5-2 mm in length and 0.3-0.4 mm in diameter. An anisotropic hydrogel material of these dimensions may shrink in length to 1-1.5 mm and will expand laterally to a diameter of 1-1.5 mm. Persons of ordinary skill in these arts will immediately recognize that the embodiments are not limited to these particular dimensions.

Stretching is preferably done after soaking of a material set forth herein, e.g., sodium gellan, carboxymethylcellulose sodium, calcium alginate or calcium gellan, in either a mineral or organic acid. Acid removes either sodium ions or cross linking cations and makes stretching far easier. Strength is relatively unaffected. The method of acidification depends upon the polymer and the extrusion solution made therefrom. If DMSO is to be used as the solvent for an extrusion bath, it is normally necessary to acidify anionic polymers before dissolution in DMSO. In this case one can use acidified water as a coagulation bath. If water is the solvent in an extrusion solution, it is preferable to extrude into aqueous solutions of metal salts before removing them by acidification. It has been found that, at least with alginate, acid coagulation baths produce weak acid gels which can be difficult to stretch.

Normally, orienting of ionically crosslinked high guluronic acid alginate, carboxymethylcellulose, and gellan is difficult and little anisotropy is achieved. Tight binding of divalent or trivalent cations results in decreased molecular mobility and is probably the main cause of poor orientation. Removal of gelling cations, however, makes the hydrogels much more plastic, so long as they do not become freely soluble in water. Therefore it is preferred that polymer carboxyl groups be acidified (protonated) rather than converted to alkali metal, tetramethylammonium, tetrabutylammonium, or ammonium salts.

Once the extrusion has been stretched, it is necessary to neutralize acid groups with metal or organic salts. This can be accomplished either in aqueous solutions or water/alcohol solutions (usually 50-70% alcohol in water). Should aqueous solutions be used, it is necessary to have highly concentrated salt—usually saturated or supersaturated—to prevent swelling and disruption of orientation. If water/alcohol solutions are used, swelling is also greatly reduced, but one must use salts soluble in alcohol. This method can be used to fabricate stretched extrusions as a mixture calcium alginate and alginic acid, at approximately an 80%:20% ratio. There will thus be less stiffness and brittleness in the final product, which should make handling easier.

An anisotropically swellable material may comprise a polysaccharide, with the polysaccharides having a substantially parallel molecular orientation relative to each other. Substantially parallel refers to a condition wherein polymers have been processed to become aligned relative to each other instead of randomly coiled. In the context of anisotropically swellable materials, an anisotropic swelling in physiological saline under non-constrained conditions is required to demonstrate substantially parallel alignment. Examples of polysaccharides include gellan, polysaccharides closely related to gellan, and polysaccharides related to gellan. The anisotropically swellable material may include an acidic polysaccharide treated with acid-catalyzed depolymerization to lower the molecular weight of the acidic polysaccharide. The anisotropically swellable material may comprise an organic or inorganic counterion or a metallic ion.

Anisotropically swellable materials were made of gellan gum. Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were dehydrated in a graded alcohol series to 91% alcohol and subsequently stretched to twice their original length. They were allowed to air dry.

The extrusions were placed into distilled water to assess neutralization, as sodium gellan, but not acidic gellan, is very soluble in distilled water. After 10 minutes the extrusions were dissolved, indicating neutralization had been achieved.

Occlusive devices were then fabricated by cutting neutralized extrusions into cylindrical pieces. Their dry dimensions were 1.524 millimeters in length and 0.254 millimeters in diameter. Once placed into physiological saline and allowed to swell to their maximum extent, they had dimensions of 1.27 millimeters in length and 1.016 millimeters in diameter.

Another set of anisotropically swellable materials were made of alginate. In another process, sodium alginate powder (15 grams) was dissolved into 100 milliliters of distilled water to make a 15% solution which was subjected to a vacuum to remove air bubbles. This solution was extruded under air pressure (45-50 pounds per square inch) into a coagulation bath of 5% calcium chloride and left to harden for 30 minutes. Extrusions were removed and washed three times in distilled water to remove unbound salt and then acidified by washing three times in 5% citric acid. Acidified alginate extrusions were again washed in distilled water and dehydrated through a graded alcohol series to 91% alcohol. Extrusions were taken from 91% alcohol and placed on a ruler to measure extent of stretching before breakage. The extrusions were found to easily be stretched to twice their original length, indicating that significant orientation could be achieved.

Dried alginic acid extrusions were placed into 5% calcium chloride in a 70% aqueous ethanol solution and allowed to incubate for two hours at which time they were removed, washed in a 70% aqueous ethanol solution for two hours, dehydrated in 91% aqueous ethanol and dried. Dried calcium alginate solutions were cut into small cylindrical pieces to simulate occlusive devices. The small pieces, 1.524 millimeters in length and 0.1905 millimeters in diameter, were placed into 0.9% sodium chloride to assess extent of swelling. After 15 minutes the dimensions were measured to be 1.27 millimeters in length and 0.508 millimeters in diameter.

Another set of anisotropically swellable materials were made of gellan gum. Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were dehydrated in a graded ethanol series and subsequently stretched to twice their length and allowed to air dry.

After drying, extrusions were placed into a 5% solution of calcium chloride in 70% aqueous ethanol and allowed to incubate for 2 hours. After rinsing in 70% aqueous ethanol for two hours and dehydration in 91% ethanol, extrusions were allowed to air dry. Dried calcium alginate extrusions were cut into small cylindrical pieces to simulate occlusive devices. The small pieces, 1.524 millimeters in length and 0.337 millimeters in diameter, were placed into 0.9% sodium chloride to assess extent of swelling. After 15 minutes their dimensions had changed to 1.27 millimeters in length and 0.762 millimeters in diameter.

Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% sodium citrate in water and allowed to incubate for 30 minutes. It was subsequently washed in 1.0% sodium chloride to remove any excess citrate ions. Extrusions were dehydrated in a graded alcohol series to 91% alcohol and subsequently stretched to twice their original length. They were allowed to air dry.

Upon drying extrusions were placed into a saturated solution of sodium tetraborate decahydrate in 70% aqueous methanol. Incubation in this medium lasted for two hours, followed by a two-hour rinse in 70% methanol and 100% methanol. After the final wash, extrusions were air dried. Dried, borate-esterified sodium gellan extrusions were cut into small cylindrical pieces to simulate occlusive devices. Their initial dimensions were 1.524 millimeters in length and 0.254 millimeters in diameter. After 15 minutes in a 0.9% sodium chloride solution, their dimensions changed to 1.27 millimeters in length and 1.016 millimeters in diameter. Borate is an effective antimicrobial. In use, the borate provides resistance to microbial attack of the polysaccharide or other material used for the device.

Chelation-Resistant Materials and Devices

Devices exposed to chelating agents during their normal use may advantageously be made from chelation-resistant materials. Chelation can have a significant effect on the physical properties of gels that are crosslinked by chelatable ions. In the case of punctum plugs, removal of ions from gels by exposure to chelating solutions, e.g., contact lens cleaners, can undesirably affect size and durability of the plug. An increase in chelation resistance enables the creation of chemically durable implants.

As set forth in greater detail in U.S. patent application Ser. No. 60/557,368, chelation-resistant (and triggerably dissoluble) ionic gels may be made using insolubilized ions. Ionic hydrogels of gellan gum, pectinic acids, alginic acids, and the like, typically can crosslink with metal ions, e.g., calcium, magnesium, zinc, copper, barium, iron, aluminum, chromium, and cerium. Metals include, e.g., alkaline earth metals, transition metals, and heavy metals. Metal ions are, in general, easily removed by chelating agents, e.g., sodium citrate or disodium EDTA, both of which are commonly found in certain medical preparations.

But metals that have been complexed with other chemicals to make a mineral are not chelatable. The introduction of a mineral-forming substance into ionic hydrogels may be used to create implants and materials that resist chelation. A mineral-forming substance may be introduced, e.g., into either a spin dope or coagulating bath used for producing these materials. Mineral-forming substances are those substances capable of forming insoluble ionic compounds with metals. Minerals are often a combination of oppositely charged substances. These include, e.g., silicates, sulfides, halides, oxides, borates, carbonates, sulfates, phosphates, arsenates, vanadates, tungstates, molybdates, hydroxides, chromates, and the like. In certain embodiments, these mineral-forming substances may be used by incorporating them so that swelling of gels is not unduly affected by the mineral phase and the mineral phase is not be removed by chelating agents. A mineral-forming substance that is reacted with an ion to form an insoluble compound is referred to as forming a mineral phase, or to create insolubilized ions.

An embodiment is a device for occluding a nasolacrimal passage, the device comprising an introducible portion that is introducible into the nasolacrimal passage to at least partially block movement of a fluid through the passage, wherein at least a part of the introducible portion comprises a polysaccharide and a mineral phase that comprises a metal. Examples of a metal in the mineral phase are calcium, magnesium, zinc, copper, barium, iron, aluminum, chromium, cerium, alkaline earth metals, transition metals, and heavy metals. The mineral phase may be a reaction product of the metal and, e.g., at least one member of the group consisting of silicates, sulfides, halides, oxides, borates, carbonates, sulfates, phosphates, arsenates, vanadates, tungstates, molybdates, hydroxides, and chromates. The degradable, chelation-resistant material may comprise a polysaccharide. Examples of polysaccharides include gellan, polysaccharides closely related to gellan, and polysaccharides related to gellan.

Punctum plugs and other nasolacrimal occlusive devices may be made with a chelation-resistant material by using the material in a mold or other process that is used to make conventional devices based on collagen or other materials. Certain embodiments include a device for occluding a nasolacrimal passage, the device including an introducible portion that is introducible into the nasolacrimal passage to at least partially block movement of a fluid through the passage, wherein at least a part of the introducible portion comprises a degradable, chelation-resistant material that is essentially completely degradable in less than about 365 days, about 180 days, about 90 days, about 7 days, or between about 1 day and about five years in vitro in a physiological saline solution kept at 37° C. Alternatively, the device can be formed to essentially last the lifetime of the patient. Persons of ordinary skill in these arts will appreciate that all ranges and values within the explicitly articulated range are contemplated.

The chelation-resistant material may further include unmineralized free metal ion-binding functional groups, so that metals may be complexed thereto, and for subsequent metal-catalyzed degradation. Chelation-resistant and triggerably dissoluble ionic gel material may include an acidic polysaccharide treated with acid-catalyzed depolymerization to lower the molecular weight of the acidic polysaccharide. The material may be anisotropically swellable, and may comprise polymers processed into an arrangement of polymers that are substantially parallel to each other. The device may be essentially completely degradable in less than about 5 days to about five years in vitro in a physiological saline solution kept at 37° C.; persons of ordinary skill in these arts will appreciate that all ranges and values between these explicit limits are contemplated, e.g., less than 7 days, 7 days, and two years. One method of using a degradable, chelation-resistant material is to facilitate its removable by exposure to salt-free water.

In one process, for example, for example, gellan gum was acidified by washing three times with 5% citric acid in water. Resulting acidified gellan powder was subsequently rinsed with water and alcohol and allowed to dry. Acidified powder (15 grams) was dissolved into 100 milliliters of dimethyl sulfoxide to make a 15% solution which was placed under vacuum to remove air bubbles. The solution was extruded under air pressure (45-50 pounds per square inch) into a 10% aqueous solution of cuprous (copper (I)) chloride. After incubation for 15-30 minutes, extrusions were thoroughly washed in deionized water, stretched, and left exposed to air. Within 1 hour extrusions took on a turquoise color indicative of oxidation of copper(I) ions to copper(II) ions. After drying was complete, extrusions were placed into physiological saline containing 0.025% disodium EDTA. Extrusions swelled to at least 100% their original size and did not lose color. If placed into 5% sodium citrate color was gradually lost over a 1 hour period, indicating that high concentrations of chelating agents are capable of binding and removing copper from this system. Low concentrations of chelating agents present in the physiological saline solution are essentially ineffective at copper removal.

In another process, for example, gellan gum was acidified by washing three times with 5% citric acid in water. Resulting acidified gellan powder was subsequently rinsed with water and alcohol and allowed to dry. Acidified powder (15 grams) was dissolved into 100 milliliters of dimethyl sulfoxide to make a 15% solution which was placed under vacuum to remove air bubbles. The solution was extruded under air pressure (45-50 pounds per square inch) into a 10% aqueous solution of ferrous (iron(II)) sulfate. After incubation for 15-30 minutes, extrusions were thoroughly washed in deionized water and placed in 100% humidity at 65° C. overnight. Upon completion of the oxidation reaction, extrusions had changed from a straw color to brown-green, indicative of oxidation of iron(II) ions to iron(III) ions. After drying, extrusions were placed into physiological saline containing 0.025% disodium EDTA. Extrusions swelled to at least 100% their original size and did not lose color. If placed into 5% sodium citrate color was gradually lost over a 1.5-2 hour period, indicating that high concentrations of chelating agents are capable of binding and removing iron from this system. Removal of iron by chelating agents was slower than was the case with copper, which is expected as copper has greater affinity for chelating ions than does iron. Low concentrations of chelating agents present in the physiological saline solution are essentially ineffective at ferric ion removal.

Controllably Degradable Materials and Devices

Some embodiments are implantable devices and materials that are made of degradable materials. Depolymerized gellan gum, depolymerized polysaccharides closely related to gellan, or depolymerized polysaccharides related to gellan gum are examples of such materials. Other polysaccharides may also be used. For example, to achieve a rapid dissolution time of 5-10 days, the molecular weight of gellan gum may be lowered. One method for lowering the molecular weight is by acid-catalyzed depolymerization. Most polysaccharides, when exposed to strong acids, will undergo hydrolysis of glycosidic bonds. This process is accelerated by heat, oxygen and/or water. And protonated uronic acid residues can also participate by catalyzing depolymerization through intramolecular catalysis. For these reasons, neutral polysaccharides typically degrade more slowly at low pH than do acid polysaccharides. Degradation of free acid forms of polysaccharides is referred to herein as autocatalytic hydrolysis. Dissolution times may thus be adjusted by controlling the amount of depolymerization, which may be performed by controlling the depolymerization conditions, e.g., heat, oxygen, and/or water. The Swellable Temporary Punctum Plug example, below, describes experiments that document how degradation can be controlled using these techniques.

Referring to FIG. 6, it is evident that the molecular weight of gellan can be very high. One method for lowering the molecular weight is with acid-catalyzed depolymerization. Most polysaccharides, when exposed to strong acids, will undergo hydrolysis of glycosidic bonds. This process is accelerated by heat, oxygen and/or water. And protonated uronic acid residues can also participate by catalyzing depolymerization through intramolecular catalysis. For these reasons, neutral polysaccharides typically degrade more slowly at low pH than do acid polysaccharides. Degradation of free acid forms of polysaccharides is referred to herein as autocatalytic hydrolysis. Dissolution times may thus be adjusted by controlling the amount of depolymerization, which may be performed by controlling the depolymerization conditions, e.g., heat, oxygen, and/or water.

Among acid polysaccharides, self-catalyzed degradation is related to the relative abundance of uronic acid residues in the polymer chain. Glycosidic linkages between uronic acid residues are more resistant to hydrolysis than are those between neutral residues. Polysaccharides composed of only uronic acid residues will thus degrade more slowly at low pH than will polysaccharides with neutral and acidic residues. Gellan possesses one uronic acid residue to every three neutral residues. It is therefore quite sensitive to autocatalytic hydrolysis. In principle, all acidic polysaccharides and their semisynthetic derivatives can be depolymerized by acidification and heat treatment with water and/or oxygen. Depolymerization would be influenced by the nature of glycosidic bonds among saccharide residues as well as the amount of uronic acid residues present in the polymer.

Autocatalytic hydrolysis can be performed at various steps in the process of preparing a material or a device. For example, gellan may be treated while in solution before forming the gellan into a material or device. Alternatively, the treatments may be performed on gellan powders, fibers, filaments and films. The only requirement is that water or oxygen should be capable of reacting with the polymer, preferably in a uniform manner so as to ensure a consistent product. Low reaction temperatures are preferred as they allow easy control over the extent of degradation. Reactions normally take 6-48 hours to complete.

Depolymerized gellan may be made that is stable in saline for 1 hour to only slightly less than that which is possible without depolymerization treatment. Similar polymers such as alginate have duration times in vivo for over 5 years, so gellan could be made with a similar durability. Durability depends on extent of polymer protonation and duration/temperature at which autocatalytic degradation proceeded. In saline, depolymerized material tends to fragment into increasingly smaller pieces. This indicates that molecular weight has been reduced via hydrolysis. In contrast, sodium gellan which has not been subjected to depolymerization is stable in saline for an indefinite time so long as it is not subjected to microbial attack.

To create a spin dope for extrusion, it has been found that solvating depolymerized gellan powder in DMSO is preferable to processing from water. Acidified gellan gum is soluble in DMSO at room temperature whereas elevated temperatures are needed to achieve 10-15% sodium gellan solutions in water. Likewise, ammonium gellan, tetramethylammonium gellan, tetrabutylammonium gellan and hydroxyethyl(trimethyl)ammonium gellan are all soluble in polar organic solvents such as DMSO but have the undesirable property of developing very high viscosities at room temperature. Heating is necessary to achieve proper solution concentration and viscosity. To avoid additional degradation which would occur if heated water or organic solvents were employed, all depolymerized gellan is acidified and processed using polar organic solvents such as DMSO. In addition, compared to water-processed gellan, DMSO-processed gellan possesses much more favorable swelling characteristics upon rewetting from a dried state.

For example, to show the creation of rapidly degradable depolymerized polymers, gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into a coagulation bath consisting of 10% citric acid in distilled water.

Extrusions were removed from the coagulation bath, washed three times in distilled water and dehydrated through a graded alcohol to series up to 91% alcohol. Once removed from 91% alcohol, extrusions were placed on a ruler, measured, and then stretched to twice their original length and allowed to dry. Once dried, extrusions were placed in an incubation chamber at 65° C. and 100% humidity for 0, 6, 8, 18 and 48 hours. Experimental groups consisted of extrusions treated at 65° C. and 100% humidity for the four time intervals; untreated extrusions acted as controls. Samples from each group were air-dried after incubation to remove excess water and then dissolved in DMSO to make a 2.5% solution. Gellan, free acid (2.5%) in DMSO from each sample group was tested for viscosity using a falling ball viscometer at 22° C. Results were as follows:

| Depolymerization Time (hours) | Solution Viscosity (centipoises) |
|---|---|
| 0 hr | 244.25 cP |
| 6 hr | 61.91 cP |
| 8 hr | 59.69 cP |
| 18 hr | 32.43 cP |
| 48 hr | 24.31 cP |

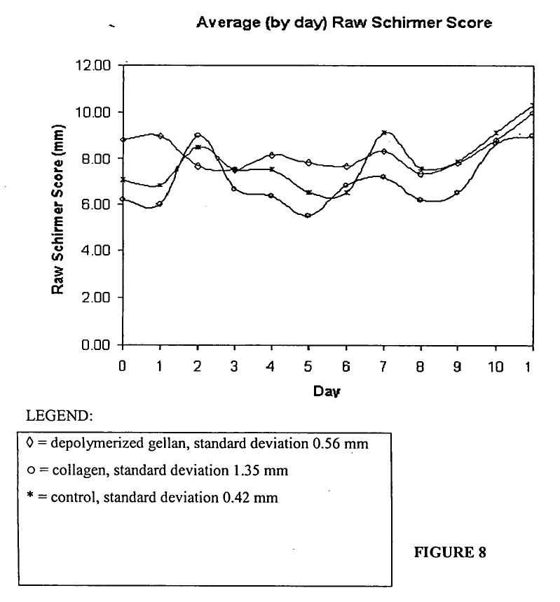

Plugs were sterilized with ethylene oxide and implanted into the nasolacrimal system of rabbits. The protocol used 12 rabbits, with the right eyes of these rabbits occluded with a temporary punctum plug, and the left eye was left unoccluded. Six days of baseline data was gathered for each rabbit, in both eyes, prior to occlusion. Six rabbits received Collagen plugs in the right eye, and the remaining six rabbits received depolymerized gellan plugs in the right eye. All left eyes were left unoccluded for the duration of the study. Each day tear film was assessed using Schirmer strip scores for both eyes, in all rabbits, and recorded as the length in millimeters of wetted strip material. The animals were also observed for any signs of irritation, epiphora, erythema, pruritus, infection, or swelling, which would indicate removal of the insert. There were no observed cases of any of these conditions in any of the animals. After the data was collected, it was analyzed in the following manner, see FIG. 8, The average daily raw Schirmer score was calculated for three different data sets, the collagen occluded eyes (six points per day), the depolymerized gellan occluded eyes (six points per day), and the unoccluded control eyes (twelve points per day). The daily standard deviation was also calculated, and averaged across all days. The daily averages were then plotted on a graph to compare the two occlusive methods with the unoccluded control group of eyes.

As is evident from the data of FIG. 8, depolymerized gellan gum can serve as a temporary plug to block the flow of fluid through an opening or duct. It performed more consistently than did the currently accepted practice of using collagen as an occlusive material.

Triggerable Dissolution of Nasolacrimal Implants

Metal-catalyzed oxidation may be used to triggerably dissolve a polymeric material. Free metal ions are associated with the polymer before, during, or after the formation of the gel. The metal ions are used as catalysts to catalyze oxidation by a peroxide, e.g., benzoyl peroxide or hydrogen peroxide, or ascorbate (vitamin C). Polymers which effectively bind metals usually have amino, carboxyl, phosphate or sulfate functional groups. Covalent or other crosslinking of such polymers to form hydrogels may therefore be accomplished so as to leave at least some functional groups free to bind metal ions. If polysaccharides are to be used to create gels, therefore, their hydroxyl groups may be utilized in crosslinking reactions instead of other groups such as carboxyls. Some or all of the polymers or materials in a gel or hydrogel may be used to capture the free metal ions. As set forth in greater detail in U.S. patent application Ser. No. 60/557,368, covalently crosslinked chelation-resistant gels for triggerable dissolution may be made by crosslinking a first polymer with a second polymer that is triggerably degradable by metal-catalyzed oxidation. Such materials may be made into a device for occluding a nasolacrimal passage as described herein, or as referenced herein.

In some embodiments, the crosslinking of a first and a second polymer may create a hydrogel, while degradation of the second polymer causes the gel to degrade. Either the first or the second polymer has functional groups that are capable of binding a metal ion. The crosslinking may be performed by, e.g., an acid-catalyzed esterification of hydroxyl and carboxyl groups. To make the gel, the first and the second polymer may be mixed together and exposed to heat under acidic conditions to crosslink their functional groups to each other or to a crosslinking agent.

Chemical removal may be effected by oxidation using peroxides (e.g., benzoyl peroxide or hydrogen peroxide) or ascorbate (vitamin C). Transition metals, especially iron and copper ions, may be used as catalysts for the reaction. In topical applications, a ferrous chloride-3% hydrogen peroxide system can be used for very rapid degradation of susceptible hydrogels. However, hydrogen peroxide typically cannot be used in the eye; therefore ferric chloride/cupric chloride-ascorbate system is advantageous. Removal of subpunctal devices may be achieved in the following manner: (1) Flush the gel with an isotonic or slightly hypertonic solution containing transition metal ions, ferric and cupric ions being preferred. The anionic groups will bind metal ions, atomically dispersed throughout the gel; (2) Rinse the surrounding tissues with neutral buffered saline or water for injection. Do not allow gels to be exposed to chelating agents such as disodium EDTA or sodium citrate; and (3) Apply diluted ascorbic acid or ascorbic acid salts to the gel. Periodic application will oxidize the gel, rendering it brittle and mechanically weak enough to crumble apart.

An embodiment is a device for occluding a nasolacrimal passage, the device comprising an introducible portion that is introducible into the nasolacrimal passage to at least partially block movement of a fluid through the passage, wherein at least a part of the introducible portion comprises at least a first polymer that is triggerably degradable by metal-catalyzed oxidation. In certain embodiments, at least a part of the introducible portion further comprises a second polymer, wherein at least one of the first and the second polymer comprises at least one functional group capable of binding a metal ion. In some cases, the first and the second polymer are crosslinked by acid-catalyzed esterification of hydroxyl and carboxyl groups. The polymers may comprise a polysaccharide, e.g., gellan, welan, S-88, S-198, a rhamsan gum. The polymers may comprise, e.g., at least one member of the group consisting of alginate, curdlan, carboxymethylcellulose, crosscarmellose, poly(acrylic acid), xanthan, carrageenan, carboxymethyl chitosan, hydroxypropyl carboxymethyl cellulose, pectin, gum Arabic, karaya gum, psyllium seed gum, carboxymethyl guar, and mesquite gum. The material may include an acidic polysaccharide treated with acid-catalyzed depolymerization to lower the molecular weight of the acidic polysaccharide. The material may comprise a metallic ion. The material may be anisotropically swellable, and may comprise polymers processed into an arrangement of polymers that are substantially parallel to each other.

As set forth in detail, herein, and in U.S. patent application Ser. No. 60/557,368, devices may be removed using metal-catalyzed oxidation. One method of removing a device for occluding a nasolacrimal passage, comprises exposing the device to metal-catalyzed oxidation to degrade a material in the device to facilitate removal of the device from the nasolacrimal passage. Such a device may have metal ion-binding functional groups to facilitate such catalytic oxidation. The device may comprise an introducible portion that is introducible into the nasolacrimal passage to at least partially block movement of a fluid through the passage, wherein at least a part of the introducible portion comprises the material.

In one embodiment, an occlusive device is removable by a metal-catalyzed oxidative processes, e.g., by exposure to a peroxide to effectively dissolve or disintegrate the device or to make the device brittle and readily subject to break-up by mechanical forces. For example, gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% ferrous sulfate in water and allowed to incubate for 30 minutes. It was subsequently washed three times in distilled water to remove any free ions. After washing extrusions were placed in an aqueous solution of 3% hydrogen peroxide. Within 1 minute the gel extrusions became very brittle and could not be manipulated with forceps without fracturing.

And, for example, gellan gum was acidified by washing three times with 5% citric acid in water. Resulting acidified gellan powder was subsequently rinsed with water and alcohol and allowed to dry. Acidified powder (15 grams) was dissolved into 100 milliliters of dimethyl sulfoxide to make a 15% solution which was placed under vacuum to remove air bubbles. The solution was extruded under air pressure (45-50 pounds per square inch) into a 10% aqueous solution of cuprous (copper (I)) chloride. After incubation for 15-30 minutes, extrusions were thoroughly washed in deionized water, stretched, and left exposed to air. Within 1 hour extrusions took on a turquoise color indicative of oxidation of copper(I) ions to copper(II) ions. Extrusions were transferred to an aqueous solution of 3% hydrogen peroxide and allowed to incubate for 1-5 minutes. When removed from the hydrogen peroxide solution, the extrusions were easily fractured as they had become embrittled. Microscopic examination at 40× revealed that the surface had become pitted with chevron-shaped crevices which were especially noticeable if attempts were made at stretching.

And, for example, sodium carboxymethylcellulose was acidified by washing three times with 5% citric acid in 70% isopropyl alcohol. The resulting acidified carboxymethylcellulose powder was subsequently rinsed with 70% isopropyl alcohol and allowed to dry. Acidified powder (15 grams) of carboxymethylcellulose was dissolved into 100 milliliters of dimethyl sulfoxide to make a 15% solution and placed under vacuum to remove air bubbles. This solution was extruded under air pressure (45-50 pounds per square inch) into 70% isopropyl alcohol acidified with 10% citric acid. After washing in progressively concentrated alcohol solutions, extrusions were stretched, dried and placed under nitrogen atmosphere and cured for 24 hours at 65° C. After curing, extrusions were placed into a 10% solution of ferric (iron(III)) chloride and allowed to incubate for 30 minutes. They were subsequently washed three times in distilled water to remove any free ions. Extrusions were then placed into dilute aqueous ascorbic acid (ca. 1-2%) and incubated for 30 minutes. After this time extrusions were stronger than were oxidized gellan extrusions but became brittle enough that fracture would occur on bending or stretching.

Fluidic Occlusive Elements and Materials

Occlusive elements may be made by introducing a fluidic material a to a space to be occluded, and allowing the material to hydrate to a more viscous condition. Production of fluid or otherwise flowable gels is straightforward. Polymers which are soluble in hot water but which gel when cooled are used. Briefly, a polymer such as gellan gum, a polysaccharide closely related to gellan, or a polysaccharide related to gellan gum is dispersed in cold water and heated until a weak solution is made. As the solution is cooling, it is beaten, stirred or otherwise vigorously agitated such that when room temperature is reached, a fluid remains. These fluids are typically non-viscous and show non-Newtonian flow. Fluid gels are then concentrated by evaporation, filtration or centrifugation until a solids content of at least about 10% is achieved. The suspension can then be extruded into a coagulating bath to form filaments. The degradation rate of these fluids may be controlled by adjusting the concentration of the polymer and the degree of mechanical agitation of the polymers.

When filaments are dried and placed in the body, they hydrate rapidly, forming a viscous fluid which resists flow. Various compositions have been made according to these methods that degrade in between 4 hours and 72 hours when implanted into an eye of a human patient. Persons of ordinary skill in these arts, after reading this disclosure, will be able to prepare such implantable compositions with a predetermined degradation time.

An embodiment is a device for occluding a nasolacrimal passage, the device comprising an aggregation of small particles introducible into the nasolacrimal passage to form a viscous suspension to at least partially block movement of a fluid through the passage. The small particles may comprise a polysaccharide. The small particles may comprise a polymer such as gellan gum, a polysaccharide closely related to gellan, or a polysaccharide related to gellan gum. The device may be essentially completely degradable in vitro in a physiological saline solution maintained at 37° C. in less than about 7, 5, 3, or 0.5 days. The aggregation may be, e.g., a filament. The device, or a portion thereof, may further comprise a therapeutic agent with/without DMSO and/or MSM. Other examples of using an occlusive nasolacrimal device are provided herein.

Materials of Water-Soluble Polymers Which Gel Under Physiological Conditions

Polysaccharides of the gellan family (gellan, welan, S-88, S-198 or rhamsan gums) can be fabricated into solid materials which imbibe water and gel in the presence of physiological fluid. In deionized water or in aqueous solutions of chaotropic agents such as tetramethylammonium chloride, no gelation occurs—the polymers remain soluble. Gelation in physiological fluids is believed to be due to the presence of sodium ions, which can act as kosmotropic agents, i.e., agents which have strong interactions with water molecules and act to maintain gel structure. Under physiological conditions, devices made with sodium gellan swell up to 3 times their original size and effectively fill spaces into which they are placed.

If left in physiological saline, sodium gellan gels will not degrade even over extended periods. Gels are nevertheless quite soluble when contacted with ion-free water. Solubility can be decreased to some extent by addition of polymers which can form hydrogen bonds with sodium gellan (polyvinyl alcohol is a primary example). This is analogous to the calcium-alginate-PVA gel system used to sequester metals or encapsulate microorganisms (Klimiuk and Kuczajowska-Zadrozna, 2002; Pattanapipitpaisal, Brown and Macaskie, 2001; Micolay et al., 2003). A factor influencing water solubility is the freedom of the gel to expand. For example, a sodium gellan gel placed unconstrained in water at room temperature will start to dissolve after 5-10 minutes. If the gel is constrained in tubing such that its lateral dimensions are fixed, it will not dissolve in ion-free water even after 24 hours. Without being committed to a specific mode of action, it is believed that constraint results in a gel concentration that is far greater than is its solubility in water.

Furthermore, it has been found that if water is injected into or around a constrained gel and is allowed to flow swiftly, sodium gellan gels will shrink in dimensions. For constrained sodium gellan gels solubility in water appears to be a function of velocity of water moving through and around the gel. Moving water is able to carry soluble polymer molecules away from the main body of the gel much more effectively than is still or slowly moving water. These results show that implants made of sodium gellan can be stable unless intentionally removed with water through irrigation. Additional details are set forth in U.S. patent application Ser. No. 60/557,368.

An embodiment is a device for occluding a nasolacrimal passage, the device including an introducible portion that is introducible into the nasolacrimal passage to at least partially block movement of a fluid through the passage, wherein at least a part of the introducible portion comprises at least one polysaccharide in the group consisting of gellan, welan, S-88, S-198 and rhamsan gum. The polysaccharide may include, e.g., an acidic polysaccharide treated with acid-catalyzed depolymerization to lower the molecular weight of the acidic polysaccharide. The polysaccharide may also include a metallic ion. The polysaccharide may also include an arrangement of polymers that are substantially parallel to each other.

Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 10% citric acid in water and allowed to incubate for 30 minutes. It was subsequently washed three times in distilled water to remove any free ions. Extrusions were dehydrated in a graded alcohol series to 91% alcohol and subsequently stretched to twice their original length. They were allowed to air dry. After drying extrusions were placed into a saturated sodium carbonate solution for 20 minutes followed by a saturated sodium chloride solution for another 20 minutes. After rinsing twice in 70% alcohol for 20 minutes each and 91% alcohol for 20 minutes, extrusions were allowed to air dry.

Extrusions were placed into distilled water to assess neutralization, as sodium gellan, but not acidic gellan, is very soluble in distilled water. After 10 minutes extrusions were dissolved, indicating neutralization had been achieved. At no time during neutralization did extrusions become soft or swell, indicating that orientation had been maintained. Prototype occlusive devices were fabricated by cutting neutralized extrusions into cylindrical pieces. Their dry dimensions were 1.524 millimeters in length and 0.254 millimeters in diameter. Once placed into physiological saline and allowed to swell to their maximum extent, they had dimensions of 1.27 millimeters in length and 1.016 millimeters in diameter.

Methods of Making Hydrophilic Extrusions, Fibers and Monofilaments Incorporating Carboxymethylcellulose

As set forth in detail in U.S. patent application Ser. No. 60/557,368, materials and devices may be made using hydrophilic extrusions, fibers and monofilaments incorporating carboxymethylcellulose. One such embodiment is a method of making a nasolacrimal implant comprising a degradable portion that comprises crosscarmellose prepared by acidification of a free acid of carboxymethylcellulose. Acidification displaces neutralizing ions (K⁺ or Na⁺), thereby causing carboxymethylcellulose to behave as a an uncharged polysaccharide such that it can be dissolved into DMSO. Dissolution in DMSO allows for much higher concentrations than is possible in water, especially if the solution is heated. The concentrated solution can then be used to fabricate extrusions in the form of fibers or monofilaments whose mechanical properties far exceed those of fibers spun from aqueous solutions. It can reasonably be expected that any acidic polysaccharide (having COOH functional groups) could be treated this way. Once the material has been shaped to its final form, here by extrusion, it can be internally crosslinked by methods already known to the arts. U.S. Pat. No. 3,379,720 discloses a method for modifying water-soluble polymers such as carboxymethylcellulose to render them insoluble in water. In the present application is disclosed a method of forming a device such as a fiber or monofilament which can then be cured to make it insoluble in water, see also U.S. Pat. No. 3,379,720.

In some embodiments, extrusions the free acid of carboxymethylcellulose are prepared from an extrusion of carboxymethylcellulose. A step may involve curing the crosscarmellose at a temperature of at least about 40° C. A step may involve acidification of a free acid of carboxymethylcellulose is performed in the presence of a polysaccharide associated with the carboxymethylcellulose. Various other features of nasolacrimal occlusive devices are set forth herein; such devices and features may be used in conjunction with embodiments related to crosscarmellose, including, e.g., drug delivery, anisotropic swelling, and various forms of degradation.

Materials of Water-Insoluble Low-Substituted Hydroxypropyl Cellulose

In some embodiments, occlusive devices are made of low substituted hydroxypropyl cellulose, which is a pharmaceutical excipient consisting of cellulose that has been reacted with propylene oxide in the presence of alkali. It is chemically identical to water-soluble hydroxypropyl cellulose except that its degree of substitution is far lower (7-16% versus 60-100%). It is insoluble in most organic solvents and is water-soluble in the presence of 10% sodium hydroxide. Highly basic solutions such as 10% sodium hydroxide can lead to depolymerization, so that the final product properties may thereby be adjusted during processing.

The chemical stability and swelling power of low substituted hydroxypropyl cellulose makes it attractive as a material for occlusive devices. Chemical stability makes it, like native cellulose, difficult to process except by the use of specialized solvents. Cellulose solvents include cuprammonium complexes dissolved in alkali, lithium chloride/N,N-dimethylacetamide, cadmium oxide/ethylenediamine and N-methylmorpholine-N-oxide. Other approaches for forming useful articles from cellulose usually involve chemical modification of cellulose molecules with easily removable functional groups. Examples include xanthanation, silylation and acetylation of cellulose followed by dissolution in alkali or organic solvents or by direct melting.

Application of the cuprammonium method to low substituted hydroxypropyl cellulose first involves formation of a copper-ammonia complex by addition of a small amount of 28-30% ammonium hydroxide to a 25% cupric sulfate solution. Precipitate formed from the reaction is insoluble in water and can be removed by simple filtration. Collected precipitate is then redissolved in 28-30% ammonium hydroxide, into which low substituted hydroxypropyl cellulose dissolves to concentrations up to 15%, with a practical limit for extrusion being about 12.5%. Once completely dissolved and deaerated under vacuum, the cuprammonium-low substituted hydroxypropyl cellulose can be extruded into acidic coagulation baths to make strong, gel-like filaments. These filaments are highly elastic and must be held in tension during the drying process to prevent rebound. Upon drying the material becomes quite strong, and its strength is maintained even upon re-hydration. Like cuprammonium rayon, cross-sectional shape is round, which is advantageous in formation of devices with consistent dimensions.

Low-substituted hydroxypropyl cellulose may also be dissolved in aqueous solutions of N-methylmorpholine oxide monohydrate under heating to 80°-110° C. and extruded into a 10% aqueous solution of ethanol. This process is known as the lyocel process which is used for processing of regenerated cellulose fibers. The use of this process results in materials with a very high degree of orientation and good mechanical properties.

Testing of occlusive devices made of low substituted hydroxypropyl cellulose has shown that dried devices swell to twice their size when placed into water or physiological saline. They do not dissolve readily in any media that would normally be encountered in medicine such as water or aqueous solutions of sodium chloride.

An embodiment involving hydroxypropyl cellulose is a biocompatible composition or a device that undergoes a transition from a first shape to a second shape when exposed to an aqueous or physiological liquid. The second shape may have a larger volume than the first shape. The device or composition may include hydroxypropyl cellulose, e.g., low substituted hydroxypropyl cellulose. Such a device may be swellable in an aqueous or physiological fluid, e.g., between 25% and 1000%, 50% and 500%, or between 100% and 400%; persons of ordinary skill in these arts will immediately appreciate that all values and ranges within these explicit ranges are contemplated.

An example of an occlusive device made of low substituted hydroxypropyl cellulose was prepared. A Cupric hydroxide was prepared by placing 125 milliliters of a 25% aqueous cupric sulfate solution into a 250 milliliter beaker and adding 13 milliliters of 30% ammonium hydroxide. Precipitated cupric hydroxide was filtered under vacuum and washed three times in cold water for 5 minutes each wash. Cupric hydroxide was then dissolved in 150 milliliters of 30% ammonium hydroxide. To this were added 18.75 grams of low-substituted hydroxypropyl cellulose under stirring. The resulting solution was held under vacuum overnight at 3° C. to remove air bubbles, then extruded through a round spinnerette under 275-310 KPa pressure into a coagulation bath consisting of 950 milliliters of 1.0N sulfuric acid and 50 milliliters of ethanol. Extruded material was allowed to harden in the coagulation bath for 20 minutes followed by rinsing three times in cold water for 5 minutes each wash. Dehydration was accomplished with a graded ethanol series and material was stretched 150% from 91% ethanol. When place in distilled water or 0.9% sodium chloride they swelled to approximately twice their original diameter and slightly decreased in length.

Drug and Therapeutic Agent Delivery

The gels and other devices set forth herein could contain medicaments, therapeutic agents, antimicrobials (e.g., silver), bioactive minerals and glasses, radioactive therapeutic materials, cytotoxic agents (for tissue ablation), etc. The gel would entrap active therapeutic agents at the site where the gel is formed in a patient, or could slowly elute therapeutic agents into the patient, e.g., into the bloodstream or other tissues. Various therapeutic agents are described in commonly owned and copending U.S. Provisional Application No. 60/550,132, entitled “Punctum Plugs, Materials, And Devices”, and may be combined with the gels and devices described herein.

Particulate silver is another agent that may be used in these gels and devices. Particulate silver exists in an aggregated or crystalline state and is essentially uncharged. Particulate silver does not interact with charged groups on polysaccharides because it does not carry a charge; as a result, particulate silver can not be a crosslinking ion that crosslinks a polysaccharide.

The therapeutic agent may be mixed with a solvent that is used to dissolve or suspend the polysaccharide; an advantage of this process is that the agent is dispersed through the solvent and is relatively well mixed into the final composition. Or the agent may be introduced into a powder of the polysaccharide. The therapeutic agent may also be introduced at other points of processing, with the choice depending on the type of agent, solvents, and eventual application.

For example, the therapeutic agent TRICLOSAN, a common antimicrobial agent, is insoluble in water but is highly soluble in DMSO and alcohols. Triclosan was added to a 15% acid gellan-DMSO solution to make a mixture of 0.5% triclosan and 15% acid gellan. The mixture was deaerated under vacuum for 2 hours to remove air bubbles and extruded under 45 psi air pressure into a coagulation bath of 2.5% sodium bicarbonate-7.5% sodium chloride. Extrusions were washed briefly in water chilled to 1-2° C. and then allowed to air dry under tension. In contrast to clear extrusions made from gellan alone, those containing triclosan appeared white. If soaked in 70% isopropyl alcohol, extrusions became clear, indicating elution of triclosan.

Another method of delivery involves exposing a material to DMSO or Methyl-sulfonyl-methane (MSM), with a therapeutic agent being contained therein. The implant, with the DMSO, MSM, or other suitable solvent still present, may be implanted. The DMSO, MSM, and/or other solvent, enhances delivery of the drug into a tissue.

Anisotropically swellable occlusive devices containing a therapeutic agent, silver, were made from gellan gum. Gellan gum was acidified by washing three times with 5% citric acid in water. Resulting acidified gellan powder was subsequently rinsed with water and alcohol and allowed to dry. Acidified powder (15 grams) of gellan gum was dissolved into 99 milliliters of dimethyl sulfoxide. A silver solution was then made by dissolution of 0.157 grams of silver nitrate in DMSO. One milliliter of this solution was added to the 99 milliliters of gellan gum solution and was subjected to a vacuum to remove air bubbles. This solution was extruded under air pressure (45-50 pounds per square inch) into 10% ascorbic acid in water and allowed to incubate for 30 minutes, at which time extrusions changed from clear and colorless to a light straw color. They were subsequently washed three times in distilled water to remove any free ions, unbound silver particles and ascorbic acid. After dehydration through a graded ethanol series extrusions were stretched to twice their original length and allowed to dry. Without being limited to a particular theory of operation, it is believed that this process results in a dispersion of silver particles throughout the hydrogel.

The anisotropically swellable occlusive devices were then produced by cutting the neutralized extrusions into cylindrical pieces. Their dry dimensions were 1.524 millimeters in length and 0.254 millimeters in diameter. Once placed into physiological saline and allowed to swell to their maximum extent, they had dimensions of 1.27 millimeters in length and 1.016 millimeters in diameter. After one week in the physiological saline solution they began to lose color and after 2-3 weeks they became clear.

Another set of anisotropically swellable devices were made with a therapeutic agent. Gellan gum was acidified by washing three times with 5% citric acid in water. Resulting acidified gellan powder was subsequently rinsed with water and alcohol and allowed to dry. Acidified powder (15 grams) of gellan gum was dissolved into 99 milliliters of dimethyl sulfoxide. A silver solution was then made by dissolution of 0.157 grams of silver nitrate in DMSO. One milliliter of this solution was added to the 99 milliliters of gellan gum solution and was subjected to a vacuum to remove air bubbles. This solution was extruded under air pressure (45-50 pounds per square inch) into 10% ascorbic acid in water and allowed to incubate for 30 minutes, at which time extrusions changed from clear and colorless to a light straw color. They were subsequently washed three times in distilled water to remove any free ions, unbound silver particles and ascorbic acid. After dehydration through a graded ethanol series extrusions were stretched to twice their original length and allowed to dry.

After drying, extrusions were placed into a 5% solution of calcium chloride in 70% aqueous ethanol and allowed to incubate for 2 hours. After rinsing in 70% aqueous ethanol for two hours and dehydration in 91% ethanol, extrusions were allowed to air dry. Occlusive devices were then fabricated by cutting calcium gellan extrusions into cylindrical pieces. Their dry dimensions were 1.524 millimeters in length and 0.254 millimeters in diameter. Once placed into physiological saline and allowed to swell to their maximum extent, they had dimensions of 1.27 millimeters in length and 0.575 millimeters in diameter. After 2-3 weeks in the distilled water they retained their original straw color.

Removal of Hydrogel Occlusive Devices by Changes in Tonicity

Swelling of hydrogels is often sensitive to changes in pH, temperature and/or tonicity. Shrinkage of gels will occur if it is subjected to an environment outside their optimal swelling conditions. This phenomenon can be used to easily flush an implanted hydrogel from its location. Or a hydrogel implant may be removed using other means after it has been forced to change its dimensions and thereby become less firmly set in place. For example, the implant may be removed by forceps, or surgically.

Changes in pH and temperature may advantageously be avoided when flushing a hydrogel implanted in the body to minimize possible tissue damage. This is especially useful in sensitive areas such as the eye or middle ear. Therefore, a safe method for changing dimensions of a hydrogel in vivo will be through alteration of tonicity. A flexible and hydrated material such as a hydrogel will collapse if exposed to steep osmotic gradients such as those imposed by hypertonic salt solutions. Very concentrated solutions of salts (for example, sodium chloride) could unfortunately irritate or damage tissues. It has been found that water soluble polymers can substitute for ionic salts to create very hypertonic solutions capable of altering (shrinking) the dimensions of hydrogel materials while remaining gentle enough to use in the body.

Preferably, the water soluble polymer used to change tonicity will be non-ionic. Polymers in this class include polyvinyl alcohol, polyethylene glycol, polyethylene oxide, etc. These can be readily dissolved at high concentration in physiological saline to create safe solutions for use in the body. Alternatively, some biocompatible polymers such as low molecular weight polyethylene glycols are liquids at room temperature; these can also be employed. Preferred polymers are those which are not only water soluble but also are lubricious in nature. Polyethylene glycol is one such example. Polysaccharide polymers are less preferred because, in general, they form very thick solutions in water, even at low concentrations.

To show feasibility of this removal method, fully swollen occlusive plugs made of sodium gellan were measured using a dissecting microscope at 40× magnification. Their dimensions were 2 mm in length and 1.5 mm in diameter. After incubation for 2.5 minutes with 40% polyethylene glycol (average molecular weight 1,000) in physiological saline, their dimensions were again measured. Length was found to be 1.5 mm and diameter was 1.0 mm. This represents a 25% decrease in length and 33% decrease in diameter. Fully swollen plugs of sodium gellan were also subjected to dehydration by glycerol. Their original dimensions were 2 mm in length and 1.5 mm in diameter. After incubation for 2.5 minutes with glycerol, their dimensions had decreased to 1.75 mm in length and 1.0 mm in diameter.

An embodiment is a method of removing a device for occluding a nasolacrimal passage, comprising exposing the device to a change in tonicity to cause a change in the size of the device to facilitate removal of the device from the nasolacrimal passage. For example, such a method may involve exposing the device to a fluid having a high osmolarity relative to physiological saline. Such a fluid may have a physiologically acceptable pH. At least one salt may be chosen to contribute to the high osmolarity of the fluid. Alternatively, or in combination with a salt, at least one polymer may be chosen to contribute to the high osmolarity of the fluid. The polymer may comprise a plurality of monomeric units having a formula of —(CH₂CH₂O)—.

An embodiment is a device comprising an introducible portion that is introducible into the nasolacrimal passage to at least partially block movement of a fluid through the passage, wherein at least a part of the introducible portion comprises at least one polysaccharide, e.g., in the group consisting of gellan, welan, S-88, S-198 and rhamsan gum.

An embodiment is a method of removing a device for occluding a nasolacrimal passage, comprising exposing the device to a change in tonicity to cause a change in the size of the device to facilitate removal of the device from the nasolacrimal passage. Hydrogels that are removable by these methods may be, e.g., degradable, anisotropically swellable, and/or processed into an arrangement of polymers that are substantially parallel to each other, and/or comprise a metallic ion.

Both pH and tonicity may be used to affect the size of a device. Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 7.5% sodium chloride and 2.5% sodium bicarbonate in water and allowed to incubate for 30 minutes. It was subsequently washed in 10% sodium chloride and then dehydrated in a graded ethanol series. After stretching and drying, they were cut into small pieces representative of an occlusive device. Dried and cut gellan extrusions were placed into physiological saline and allowed to swell to maximum size, which was measured using a dissecting microscope at 40× magnification. Their dimensions were 2 mm in length and 1.5 mm in diameter. After incubation for 2.5 minutes with 40% polyethylene glycol (average molecular weight 1,000) in physiological saline, their dimensions were again measured. Length was found to be 1.5 mm and diameter was 1.0 mm. This represents a 25% decrease in length and 33% decrease in diameter.

Occlusive devices may be shrunk using changes in pH, tonicity, or a combination applied at different times. Gellan gum was acidified by washing three times with 5% citric acid in water. Resulting acidified gellan powder was subsequently rinsed with water and alcohol and allowed to dry. Acidified powder (15 grams) of gellan gum was dissolved into 100 milliliters of dimethyl sulfoxide to make a 15% solution which was subjected to a vacuum to remove air bubbles. This solution was extruded under air pressure (45-50 pounds per square inch) into 7.5% sodium chloride and 2.5% sodium bicarbonate in water and allowed to incubate for 30 minutes. It was subsequently washed in 10% sodium chloride and then dehydrated in a graded ethanol series. After stretching and drying, they were cut into small pieces representative of an occlusive device. Dried and cut gellan extrusions were placed into physiological saline and allowed to swell to maximum size, which was measured using a dissecting microscope at 40× magnification. Their dimensions were 2 mm in length and 1.5 mm in diameter. The fully swollen plugs of sodium gellan were then subjected to dehydration by pure glycerol. After incubation for 2.5 minutes with glycerol, their dimensions had decreased to 1.75 mm in length and 1.0 mm in diameter. This represents a 12.5% decrease in length and a 33% decrease in diameter.

Additional Embodiments

The materials described herein may be made into a device with a predetermined structure suitable for its intended use. A predetermined structure has a shape that is determined prior to introduction into a patient. For example, a polysaccharide hydrogel formed into a punctum plug shape for use as a punctum plug has a predetermined shape. In contrast, a polysaccharide sprayed onto a tissue or injected as a liquid into a tissue does not have a predetermined shape; instead, the materials are merely provided in any convenient form for delivery to the site. Thus, e.g., plugs, tampons, packing strips, sheets, particles, spheres, blocks, cubes, cylinders, and cones, are all contemplated as particular predetermined shapes. For example, packing made of a polysaccharide may be made for packing into a nasal or sinus cavity for treating patients that have undergone sinus surgeries. Or a stuffing may be made to fill a wound created surgically or by an accident. Or particles may be made to serve as a packing material, with large particles beings suitable for large wounds and microparticles being suited for smaller embolic applications or some minimally invasive surgeries requiring delivery by a catheter, e.g., with the microparticles having a maximum cross-sectional area of between about 1-10,000 square microns, e.g., a 100×100 micron cross-sectional area. Or, for example, strips provided, e.g., from a roll or other dispenser, with a thickness of between about 0.5 mm and about 5 mm may conveniently be used for packing a wound or lumen or void, e.g., a sinus cavity.

Another embodiment is a punctum plug for blocking the flow of lacrimal fluid in an eye, comprising a shaft having a first end and a second end and sized to fit within the punctal opening, said shaft being formed from a dehydrated hydratable material having a hydrated size which is at least, e.g., one, two, three, or four times its dehydrated size, or between one and ten times its hydrated size; persons of skill in these arts will immediately recognize that all values and ranges within the explicitly stated ranges are contemplated. Such a device may further comprise a head connected to said first end of said shaft and formed substantially as a dome from said dehydrated hydratable material. A tip may be connected to said second end of said shaft and formed from said dehydrated hydratable material. The tip may be formed as a frustrum. The material for forming at least a portion of such a device maybe a material as set forth elsewhere herein. The shaft may have a tapered second end. The shaft may defines an axial bore from said first end of said shaft toward said second end of said shaft, but not through said second end of said shaft. The head may define an axial bore through said head. The shaft may be formed having two frustra-conical sections, a first frustrum near said first end and a second frustrum near said second end, said first frustrum narrowing as it tapers from said first end toward said second end, said second frustrum narrowing as it tapers from said second end toward said first end.

Another embodiment is a method of self-inserting a self-insertable punctum plug formed from a dehydrated hydratable biocompatible material, comprising: a) obtaining an insertion tool and a dehydrated hydratable punctum plug; b) fitting the insertion tool with a proximal end of the punctum plug; c) holding the insertion tool with the first hand of the recipient; d) retracting a bottom lid of an eye with a second hand of a recipient such that a punctal opening of the recipient is exposed; e) moving said insertion tool such that a distal end of said punctum plug is directed toward said punctal opening of said recipient; f) inserting said punctum plug through said punctal opening of the recipient g) releasing said punctum plug from said insertion tool; and h) releasing said bottom lid of said eye. The inserting may comprise inserting until a head of said punctum plug is positioned adjacent the punctal opening of said recipient.

Another embodiment is a method of forming a self-insertable punctum plug formed from a dehydrated hydratable biocompatible material, comprising: a) selecting a dehydrated hydratable biocompatible material which when hydrated at least doubles in size; and b) forming a punctum plug from said dehydrated hydratable biocompatible material with a shaft and a head which has a diameter greater than the shaft, said shaft formed to be of a size to fit within the punctual opening such that when said dehydrated material is hydrated, said punctum plug is sized to fit securely within the punctal opening.

Another embodiment is a nasolacrimal occlusive device that comprises gellan and is degradable in a nasolacrimal canaliculus in about 20-40 days, e.g., about 30 days. Such a device may be swellable in a physiological fluid, e.g., between 25% and 1000%, 50% and 500%, or between 100% and 400%; persons of ordinary skill in these arts will immediately appreciate that all values and ranges within these explicit ranges are contemplated.

Swellable Temporary Punctum Plugs

A series of swellable temporary punctum plugs have been made that embody many of the inventions described herein. A swellable temporary punctum plug may be designed to sit beyond the punctal ring, and can be removed in one of several ways. It may be irrigated with saline solution, it can be palpated after hydration to break the plug into pieces so it can be passed through the lacrimal system or upward through the punctum, it can be probed out with a lacrimal probe, or it may be left in place to dissolve, e.g., within 30 days of insertion. The swellable temporary punctum plug may be designed to completely dissolve within 30 days, and move out of the lacrimal system via the nasolacrimal duct. It is then expelled through the nasal cavity or into the stomach where it is ingested and passed through the excretory system. Swellable temporary punctum plugs can be made to have no sharp edges after they are hydrated, with the shape of the plug conforming to the volume that constrains it. This feature serves to limit any foreign body reaction, and the short duration serves to limit any infection that may occur. Swellable temporary punctum plugs have been made that generally take 5-10 minutes to become fully hydrated by the action of tear production, or by the use of saline drops if tear volume is not sufficient (as may be expected from patients suffering from dry eye).

In Vitro Testing of Gellan, Depolymerized to Varying Degrees

To simulate how depolymerized sodium gellan extrusions would behave when inserted into the lacrimal system, five extrusions from each of the five experimental groups were placed into clear silicone tubing with an inside diameter (ID) of 0.5 mm (0.020″). Another five were allowed to swell unconstrained. Therefore, for each experimental group there were 10 samples. The experimental groups were determined by the amount of time, in hours, heat and humidity depolymerization was applied to the gellan.

The gellan extrusions were approximately 0.3 mm (0.012″) in diameter when dried, and swelled to 1.5 mm (0.059″) when exposed to a sterile saline solution (Sight Savers, Inc. Greenville, S.C.), if unconstrained. Swelling to maximum size was complete after 15 minutes. FIGS. 7A and 7B shows swellable temporary punctum plugs designed to sit below the punctal sphincter to occlude the lacrimal system and keep the temporary plug from being spontaneously extruded through the punctal opening. The temporary plug expands laterally upon insertion via hydration by the patient's tears. The intracanalicular soft tissue is pliable and conforms to the shape of the hydrated, expanded Swellable Temporary Punctum Plug. The initial dimensions of one such plug (before insertion) are 0.3 mm in diameter, and 1.5 mm in length. After insertion, it hydrates, and expands to fill the intracanalicular space, with a maximum hydrated size of 1.5 mm in diameter and 1.25 mm in length.

Gellan gum that has been depolymerized by action of heat plus humidity, will begin to crumble after immersion in saline. This was especially noticeable if extrusions are subject to manipulation, as with the hands or with forceps.

Therefore, in order to assess durability in vitro, at given times unconstrained extrusions were picked up from the saline solution. If they remained intact, they were considered effective. If they were too weak to be handled without crumbling, they were considered as ineffective. If the extrusion is crumbled in vivo, due to the peristaltic action of the lacrimal system, it would be ineffective. Extrusions which were constrained in clear silicone tubing were gently stretched and/or bent to assess effectiveness. Movement of the tubing, and direct visualization of the gellan plug, easily revealed whether the material inside was intact, or an aggregation of pieces of a broken gel.

TABLE 2
Results of gellan dissolution from in vitro bench testing

| Experimental group (by | | | |
|---|---|---|---|
| hours of | | | |
| depolymerization) | Constraint | Days of effectiveness | |
| 0 hour | Unconstrained | >30 | days |
| 0 hour | Constrained | >30 | days |
| 6 hour | Unconstrained | 6-7 | days |
| 6 hour | Constrained | 7-10 | days |
| 8 hour | Unconstrained | 6-7 | days |
| 8 hour | Constrained | 7-10 | days |
| 18 hour | Unconstrained | 1-2 | days |
| 18 hour | Constrained | 3-4 | days |
| 48 hour | Unconstrained | <1 | day |
| 48 hour | Constrained | <1 | day |

Based on these data, a correlation between dissolution time (duration) and depolymerization time can be made, and a predictable, verifiable set of production parameters may be used to optimize dissolution time for a particular device.

Swellable temporary punctum plugs may include a length of rigid, hydrophilic, dissolvable material. Common forceps Oewelers, collagen or otherwise) may be used in the insertion of swellable temporary punctum plugs.

All patents, patent applications, and publications set forth herein are hereby incorporated by reference herein. The headings, while placed for general convenience of the reader, are not intended to limit the embodiments.

1. A punctum plug for blocking flow of lacrimal fluid in an eye comprising: an introducible portion of the plug sized for introduction into a punctal opening of the eye, the portion comprising a dehydrated material hydratable by physiological saline to swell from a first diameter to a second diameter that is at least 50% greater than the first diameter, wherein the portion is swellable by the lacrimal fluid to occlude the punctal opening to block the flow of the lacrimal fluid through the punctal opening, wherein the dehydratable material degrades in less than about seven days in the punctal opening of the patient.

2. The plug of claim 1, wherein the second diameter is at least twice the first diameter.

3. The plug of claim 1, wherein the introducible portion is a shaft.

4. The plug of claim 1, wherein the shaft further comprises a ridge or a collapsible portion.

5. The plug of claim 4, wherein the shaft is connected to a head formed substantially as a dome.

6. The plug of claim 1, wherein the dehydrated material is partially dehydrated prior to introduction into the punctal opening of the eye.

7. The plug of claim 1, further comprising a member that is sized to remain outside the punctal opening of the eye.

8. The plug of claim 1, wherein the dehydrated material comprises a polysaccharide.

9. The plug of claim 8, wherein the dehydrated material comprises a member of the group consisting of gellan, welan, S-88, S-198, and a rhamsan gum.

10. The plug of claim 1, wherein the dehydrated material comprises a member of the group consisting of alginate, curdlan, carboxymethylcellulose, crosscarmellose, poly(acrylic acid), xanthan, carrageenan, carboxymethyl chitosan, hydroxypropyl carboxymethyl cellulose, pectin, gum Arabic, karaya gum, psyllium seed gum, carboxymethyl guar, and mesquite gum.

11. The plug of claim 1, wherein the plug comprises a therapeutic agent for release from the plug after contact with the lacrimal fluid.

12. The plug of claim 1, wherein the plug comprises a preservative agent, an antimicrobial agent, an agent that is both a preservative and an antimicrobial, or a combination thereof.

13. The plug of claim 1, wherein the plug comprises silver.

14. The plug of claim 1, wherein the plug is comprised of anionic polymers crosslinked by an insoluble metal salt.

15. A method of occluding a punctal opening of an eye comprising: introducing an introducible portion of a punctum plug into the punctal opening of the eye, the introducible portion comprising a dehydrated material hydratable by physiological saline to swell from a first diameter to a second diameter that is at least 50% greater than the first diameter, wherein the portion is swellable by the lacrimal fluid to occlude the punctal opening to block the flow of the lacrimal fluid.

16. The method of claim 15, further comprising inserting said introducible portion through the punctal opening until a head of the punctum plug is positioned outside and adjacent to the punctal opening.

17. The method of claim 15, wherein the second diameter is at least twice the first diameter.

18. The method of claim 15, wherein the dehydrated material is partially dehydrated prior to introduction into the punctal opening of the eye.

19. The method of claim 15, wherein the dehydrated material comprises a polysaccharide.

20. The method of claim 19, wherein the dehydrated material comprises a member of the group consisting of gellan, welan, S-88, S-198, and a rhamsan gum.

21. The method of claim 15, wherein the plug further comprises a therapeutic agent for release from the plug after contact with the lacrimal fluid.

22. The method of claim 15, wherein the plug comprises a preservative agent, an antimicrobial agent, an agent that is both a preservative and an antimicrobial, or a combination thereof.

23. The method of claim 15, wherein the plug comprises silver.

24. The method of claim 15, wherein the plug is comprised of anionic polymers crosslinked by an insoluble metal salt.

25. A device for occluding a nasolacrimal passage, the device comprising an introducible portion that is introducible into the nasolacrimal passage to at least partially block movement of a fluid through the passage, wherein the introducible portion comprises an anisotropically swellable material that anisotropically swells in vitro in a physiological saline solution when not subjected to constraining forces.

26. The device of claim 25, wherein the anisotropically swellable material comprises a volume, a first length and a second length perpendicular to the first length, wherein exposure to physiological fluid causes the volume to increase, the first length to undergo a first percentage increase and the second length to undergo a second percentage increase that is less than the first percentage increase for the first length.

27. The device of clam 26, wherein the first percentage increase is at least 100%.

28. The device of claim 26, wherein the second percentage increase is less than 0%.

29. The device of claim 26, wherein the first length is structured to swell against a wall of the nasolacrimal passage when disposed in the nasolacrimal passage.

30. The device of claim 25, wherein the device further comprises a shaft and a head at a proximal end of the shaft, with the shaft comprising the introducible portion.

31. The device of claim 25, wherein the anisotropically swellable material comprises polymers processed into an arrangement of polymers that are substantially parallel to each other.

32. The device of claim 25, wherein the anisotropically swellable material comprises a polysaccharide.

33. The device of claim 32, further comprising copper or iron associated with the polysaccharide.

34. The device of claim 25, wherein the anisotropically swellable material comprises at least one polysaccharide chosen from the group consisting of gellan, welan, S-88, S-198, and a rhamsan gum.

35. The device of claim 25, wherein the anisotropically swellable material comprises an acidic polysaccharide or salt thereof depolymerized to lower the molecular weight of the acidic polysaccharide.

36. The device of claim 25, further comprising a degradable portion.

37. The device of claim 25, further comprising a therapeutic agent.

38. The device of claim 25, wherein the plug further comprises a preservative agent, an antimicrobial agent, an agent that is both a preservative and an antimicrobial, or a combination thereof.

39. The device of claim 25 further comprises a metal and the plug is degradable by metal-catalyzed oxidation using an oxidation agent.

40. The device of claim 25, wherein the device comprises silver.

41. The device of claim 25, wherein the device comprises borate.

42. The device of claim 25, wherein the introducible portion is comprised of anionic polymers crosslinked by an insoluble metal salt.

43. A method of occluding a nasolacrimal passage, the method comprising introducing an occlusive device into the passage, wherein the device comprises an introducible portion that is introducible into the passage to at least partially block movement of a fluid through the passage, wherein the introducible portion comprises an anisotropically swellable material that anisotropically swells in vitro in a physiological saline solution when not subjected to constraining forces.

44. The method of claim 43, further comprising using the device to treat at least one eye in a patient having at least one condition chosen from the group consisting of dry eye, seasonal allergy, and trauma caused by surgical correction.

45. A method of making a nasolacrimal occlusive device that includes a polymeric material made of polymers, the method comprising: aligning the polymers of the polymeric material in a predominantly parallel orientation relative to each other.

46. The method of claim 45, wherein aligning the polymers comprises at least one technique chosen from the group consisting of spin coating, spray coating, stretching, unidirectional freezing, extrusion from liquid crystalline solution, ordered convection, and stretching plus drying of an extrusion.

47. The method of claim 45, wherein aligning the polymers comprises stretching the material.

48. The method of 47, further comprising soaking the polymeric material in a fluid comprising mineral acids, organic acids or salts of monovalent cations before stretching the polymeric material.

49. The method of claim 45, wherein the polymeric material comprises at least one member of the group consisting of gellan gum and its salts, carboxymethylcellulose and its salts and alginic acid and its salts.

50. The method of claim 45, wherein aligning the polymers comprises acidification of anionic polymers before dissolution in an organic solvent.

51. The method of claim 45, wherein the device comprises silver or borate.

52. The method of claim 45, wherein the introducible portion is comprised of anionic polymers crosslinked by an insoluble metal salt.

53. A device for occluding a nasolacrimal passage, the device comprising: an introducible portion that is introducible into the nasolacrimal passage having a structure to at least partially block movement of a fluid through the passage, wherein at least a part of the introducible portion comprises at least one polysaccharide in the group consisting of gellan, welan, S-88, S-198 and rhamsan gum.

54. The device of claim 53, wherein the device further comprises a shaft and a head at a proximal end of the shaft, with the shaft comprising the introducible portion.

55. The device of claim 53, wherein the polysaccharide comprises an acidic polysaccharide or salts thereof depolymerized to lower the molecular weight of the acidic polysaccharide.

56. The device of claim 53, further comprising copper or iron associated with the polysaccharide.

57. The device of claim 53, wherein the polysaccharide comprises polymers processed into an arrangement of polymers that are substantially parallel to each other.

58. The device of claim 53, further comprising a therapeutic agent.

59. The device of claim 53, further comprising a preservative agent, an antimicrobial agent, an agent that is both a preservative and an antimicrobial, or a combination thereof.

60. The device of claim 53, further comprising a borate ester.

61. The device of claim 53, wherein the device is essentially completely degradable in less than about 7 days in vitro in a physiological saline solution kept at 37° C.

62. The device of claim 61, wherein the introducible portion is the entire device.

63. The device of claim 53, wherein the device comprises silver.

64. The device of claim 53, wherein the introducible portion is comprised of anionic polymers crosslinked by an insoluble metal salt.

65. A method of occluding a punctal opening of an eye comprising: introducing an introducible portion of a punctum plug into the punctal opening of the eye, the introducible portion comprising wherein at least a part of the introducible portion comprises at least one polysaccharide in the group consisting of gellan, welan, S-88, S-198 and rhamsan gum.

66. The method of claim 65, wherein the device further comprises a shaft and a head at a proximal end of the shaft, with the shaft comprising the introducible portion.

67. The method of claim 65, wherein the polysaccharide comprises an acidic polysaccharide or salt thereof depolymerized to lower the molecular weight of the acidic polysaccharide.

68. The method of claim 65, further comprising copper or iron associated with the polysaccharide.

69. The method of claim 65, wherein the polysaccharide comprises polymers processed into an arrangement of polymers that are substantially parallel to each other.

70. The method of claim 65, with the plug further comprising a therapeutic agent.

71. The method of claim 65, wherein the plug further comprises a preservative agent, an antimicrobial agent, an agent that is both a preservative and an antimicrobial, or a combination thereof.

72. The method of claim 65, wherein the plug further comprises a metal and the plug is degradable by metal-catalyzed oxidation using an oxidation agent.

73. The method of claim 65, further comprising shrinking the plug by exposure to hypertonic solutions.

74. The method of claim 65, wherein the plug further comprises borate.

75. The method of claim 65, wherein the device is essentially completely degradable in less than about 7 days in vitro in a physiological saline solution kept at 37° C.

76. The method of claim 65, wherein the introducible portion is the entire device.

77. The method of claim 65, further comprising using the device for treatment of at least one eye in a patient having at least one condition chosen from the group consisting of dry eye, seasonal allergy, and trauma caused by surgical correction.

78. The method of claim 65, wherein the device comprises silver.

79. The method of claim 65, wherein the introducible portion is comprised of anionic polymers crosslinked by an insoluble metal salt.
